(19) United States
(12) Patent Application Publication
(10) Pub. No.: US 2018/0092627 A1
(43) Pub. Date: Apr. 5, 2018

(54) ULTRASOUND SIGNAL PROCESSING DEVICE, ULTRASOUND SIGNAL PROCESSING METHOD, AND ULTRASOUND DIAGNOSTIC DEVICE

(71) Applicant: Konica Minolta, Inc., Tokyo (JP)

(72) Inventor: Yasuaki Susumu, Osaka-shi (JP)

(73) Assignee: Konica Minolta, Inc., Tokyo (JP)

(21) Appl. No.: 15/724,418

(22) Filed: Oct. 4, 2017

(30) Foreign Application Priority Data
Oct. 4, 2016 (JP) 2016-196787

(51) Int. Cl. A61B 8/00; A61B 8/08

(52) U.S. Cl. CPC A61B 8/4494; A61B 8/5207; A61B 8/14; A61B 8/5246; A61B 8/4272

(57) ABSTRACT

An ultrasound signal processing device includes: a receiver acquiring a receive signal sequence based on reflected detection waves received in time sequence from a subject, to generate receive signal frame data in a first orthogonal space (time direction and azimuth direction); an orthogonal space transform unit transforming the receive signal frame data to a second orthogonal space, to generate observed spectrum frame data; a transform processor processing observed spectrum partial frame data corresponding to a partial region in the second orthogonal space of the observed spectrum frame data, to generate transformed spectrum partial frame data in a third orthogonal space; and an orthogonal space inverse transform unit performing an inverse orthogonal transform on the transformed spectrum partial frame data to an orthogonal space (subject depth direction and the azimuth direction), to generate acoustic line signals for observation points in a region of interest, to generate acoustic line signal frame data.

Japanese Patent Application No. 2016-196787 filed on Oct. 4, 2016, including description, claims, drawings, and abstract, is incorporated herein by reference in its entirety.

### BACKGROUND

### Technical Field

The present disclosure relates to ultrasound signal processing devices, ultrasound signal processing methods, and ultrasound diagnostic devices provided with same, and in particular to signal beamforming processing methods in ultrasound signal processing devices that use blood flow measurement, color flow mapping, and tissue elasticity measurement.

### Description of Related Art

An ultrasound diagnostic device transmits ultrasound into a subject via an ultrasound probe (hereinafter, “probe”), and receives reflected ultrasound (echoes) that occur due to differences in acoustic impedance of tissues in the subject. Further, based on electrical signals derived from the received signals, the ultrasound diagnostic device generates an image showing structure of tissues in the subject, and displays the image on a monitor (hereinafter, “display”). Ultrasound diagnostic devices are widely used for morphological diagnosis of living bodies because they are not very invasive and allow observation of the state of internal tissues in real-time via tomographic images and the like.

Recently, ultrasound diagnostic devices are being provided with a color flow mapping (CFM) method. According to the CFM method, a Doppler shift (frequency deviation) occurring in an echo due to movement of body tissue such as blood flow is detected from a phase difference between a transmitted wave and a reflected wave, and velocity information in the form of a two-dimensional image is superimposed on a two-dimensional image (B mode tomographic image). In order to detect Doppler shift, it is necessary to repeatedly transmit and receive ultrasound to the same position in the subject (hereinafter, the number of times ultrasound is transmitted and received from the same position is called the “ensemble” and an image generated by the CFM method is referred to as a “color Doppler image”). Thus, in the CFM method, an amount of computation per unit time in reception beamforming processing is large, and a configuration is adopted that transmits a plane wave that does not have a focal point at which a reflected wave is obtained from an entire analysis target range in the subject from one transmission and reception.

However, recently, regarding the CFM method, a further increase in ensemble number to obtain image quality close to real-time processing is sought, and therefore it has become necessary to further reduce the amount of calculation in reception beamforming processing. Thus, for example, in “‘Stolt's f-k Migration for Plane Wave Ultrasound Imaging’, D. Garcia, L. Tarnec, S. Muth, E. Montagnon, J. Poree, and G. Cloutier, IEEE Transactions on Ultrasonics, Ferroelectrics, and Frequency Control, vol. 60, no. 9, September 2013”, a technique is considered of transmitting a plane wave and performing beamforming in a frequency range domain (frequency-wavenumber (f-k) domain) of the obtained reflected wave.

### SUMMARY

However, with the technique described in “Stolt's f-k Migration for Plane Wave Ultrasound Imaging,” when frame rate is to be further improved for real-time processing in the CFM method, the reduction in calculation amount is not sufficient.

In one or more embodiments of the invention, the amount of calculation in reception beamforming of a reflected wave obtained from ultrasound transmission to a subject is reduced.

An ultrasound signal processing device reflecting one aspect of the present invention is connectable to a probe in which transducers are arranged in a row, the ultrasound signal processing device including ultrasound signal processing circuitry, the ultrasound signal processing circuitry comprising: a transmission beamformer that supplies detection wave pulses to the transducers that cause the transducers to transmit detection waves that pass through at least a region of interest that represents a range to be analyzed in a subject; a reception beamformer that generates acoustic line signal frame data for observation points in the region of interest, based on reflected detection waves reflected from subject tissue and received in a time sequence by the transducers, the reflected detection waves corresponding to detection waves transmitted; and an image generator that generates ultrasound image frame data from the acoustic line signal frame data, wherein the reception beamformer includes: a receiver that acquires, for each transducer, a receive signal sequence based on reflected detection waves received in a time sequence from the subject, to generate receive signal frame data in a first orthogonal space defined by a time direction and a transducer row direction; an orthogonal space transform unit that transforms the receive signal frame data from the first orthogonal space to a second orthogonal space, to generate observed spectrum frame data; a transform processor that performs predefined calculation processing on observed spectrum partial frame data corresponding to a range in the second orthogonal space of the observed spectrum frame data, to generate transformed spectrum partial frame data in a third orthogonal space; and an orthogonal space inverse transform unit that performs an inverse orthogonal transform on the transformed spectrum partial frame data to an orthogonal space defined by a subject depth direction and the transducer row direction, to generate acoustic line signals for the observation points in the region of interest, in order to generate the acoustic line signal frame data.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the invention. In the drawings:

FIG. 1 is a function block diagram of ultrasound diagnostic system 1000 pertaining to Embodiment 1;

FIG. 2A and FIG. 2B are schematic diagrams showing detection waves;

FIG. 3 is a function block diagram showing a configuration of reception beamformer 104 pertaining to Embodiment 1;

FIG. 4 is a function block diagram showing a configuration of orthogonal space reception beamforming unit 1041 pertaining to Embodiment 1;

FIG. 5 is a function block diagram showing a configuration of CFM processor 105 and image former 107 pertaining to Embodiment 1;

FIG. 6 is a flowchart showing operations of ultrasound diagnostic device 100 pertaining to Embodiment 1;

FIG. 7 is a flowchart showing an overview of reception beamforming operations pertaining to Embodiment 1;

FIG. 8 is a schematic diagram showing aspects of frame data and partial frame data obtained by reception beamforming operations pertaining to Embodiment 1;

FIG. 9 is a schematic diagram showing a relationship between a propagation wave p and a wavenumber vector in the subject;

FIG. 10 is a schematic diagram showing a transformation from a second orthogonal space (ω, κx) to a third orthogonal space (κx,κz);

FIG. 11 is a flowchart showing detail of reception beamforming operations pertaining to Embodiment 1;

FIG. 12 is a function block diagram showing a configuration of orthogonal space reception beamforming unit 1041 pertaining to Embodiment 2;

FIG. 13 is a flowchart showing an overview of reception beamforming operations pertaining to Embodiment 2;

FIG. 14 is an explanatory diagram showing ranges of observed spectrum frame data P0, which are processing target ranges in reception beamforming pertaining to Embodiment 2;

FIG. 15 is a function block diagram showing a configuration of orthogonal space reception beamforming unit 1041 pertaining to Embodiment 3;

FIG. 16 is a flowchart showing an overview of reception beamforming operations pertaining to Embodiment 3;

FIG. 17 is a flowchart showing detail of an operation of acoustic line signal calculation processing in step S170B;

FIG. 18 is a schematic diagram showing aspects of frame data and partial frame data obtained by reception beamforming operations pertaining to Embodiment 3;

FIG. 19 is a function block diagram showing a configuration of orthogonal space reception beamforming unit 1041 pertaining to Embodiment 4;

FIG. 20 is a flowchart showing an overview of reception beamforming operations pertaining to Embodiment 4;

FIG. 21 is a flowchart showing detail of an operation of observed spectrum calculation processing in step S130C; and

FIG. 22 is a schematic diagram showing aspects of frame data and partial frame data obtained by reception beamforming operations pertaining to Embodiment 4.

### DETAILED DESCRIPTION OF EMBODIMENTS

### Embodiment 1

### <Ultrasound Diagnostic System 1000>

The following is a description of an ultrasound diagnostic device 100 pertaining to Embodiment 1, with reference to the drawings.

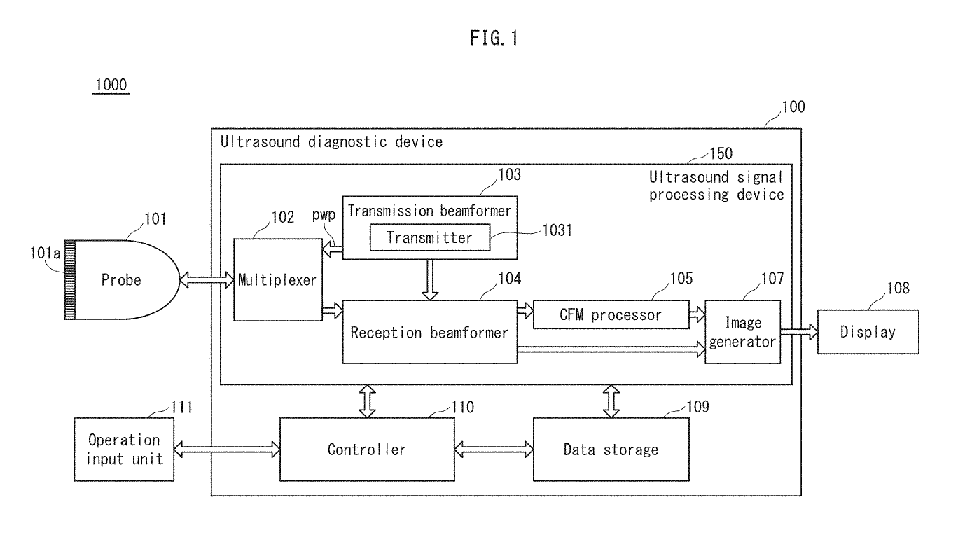

FIG. 1 is a function block diagram of an ultrasound diagnostic system 1000 pertaining to Embodiment 1.

As shown in FIG. 1, the ultrasound diagnostic system 1000 includes a probe 101 that has a plurality of transducers 101a for transmitting ultrasound into a subject and receiving waves reflected therefrom, an ultrasound diagnostic device 100 that causes the probe 101 to transmit and receive detection waves in order to generate ultrasound images based on output signals from the probe 101, and a display 108 that displays the ultrasound images on a screen. The probe 101, the display 108, and an operation input unit 111 are each connectable to the ultrasound diagnostic device 100. FIG. 1 shows the probe 101, the display 108, and the operation input unit 111 connected to the ultrasound diagnostic device 100. The probe 101, the display 108, and the operation input unit 111 may be provided inside the ultrasound diagnostic device 100.

The following describes each element externally connected to the ultrasound diagnostic device 100.

The probe 101 includes a transducer array (101a) made from a plurality of transducers 101a arranged in a one-dimensional direction (hereinafter, “azimuth direction”) representing a transducer row direction. The probe 101 converts a pulsed electrical signal supplied from a transmission beamformer 103, described later, to pulsed ultrasound. The probe 101, while transducer outer surfaces of the probe 101 are in contact with a skin surface of the subject via ultrasound gel or the like, transmits an ultrasound beam composed of a plurality of ultrasound waves emitted from a plurality of transducers towards a measurement object. The probe 101 then receives reflected waves from the subject, converts these reflected waves into electric signals via a plurality of transducers 101a, and supplies the electric signals to a reception beamformer 104.

The operation input unit 111 receives various operation inputs such as settings and operations with respect to the ultrasound diagnostic device 100 from a user, and outputs to a controller 110.

The operation input unit 111 may be, for example, a touch panel integrated with the display 108. In this case, various settings and operations of the ultrasound diagnostic device 100 can be performed by touch operations and drag operations on operation keys displayed on the display 108, and the ultrasound diagnostic device 100 is configured to be operable via the touch panel. Alternatively, the operation input unit 111 may, for example, be a keyboard that has various operation keys or an operation panel that has a means of operation such as buttons and a lever. Further, the operation input unit 111 may be a mouse or similar for moving a cursor displayed on the display 108. Further, the operation input unit 111 may be a combination of some or all of these examples.

The display 108 is a display device for image display, and displays an image output from an image generator 107 (described later) to a screen. A liquid crystal display, a cathode ray tube (CRT), an organic electroluminescence (EL) display, and the like can be used for the display 108.

### <Ultrasound Diagnostic Device 100>

The ultrasound diagnostic device 100 includes a multiplexer 102, the transmission beamformer 103 and the reception beamformer 103. The multiplexer 102 selects each transducer to be used in transmission and/or reception, among the transducers 101a of the probe 101, and secures input/output for selected transducers. The transmission beamformer 103 controls timing of application of high voltage to each of the transducers 101a of the probe 101 in order to transmit a detection wave. The reception beamformer 104 amplifies electrical signals obtained by a plurality of the transducers 101a based on reflected detection waves received by the probe 101, performs analog-to-digital (A/D) conversion, and performs reception beamforming to generate an acoustic line signal. The ultrasound diagnostic device 100 further includes a CFM processor 105, the image generator 107, a data storage 109, and the controller 110. The CFM processor 105 performs a frequency analysis of output signals from the reception beamformer 104 to generate color flow information. The image generator 107 converts acoustic line signal frame data output from the reception beamformer 104 to a tomographic image (B mode image) and overlays this with color flow information to generate a color Doppler image and displays same on the display 108. The data storage 109 stores acoustic line signals output by the reception beamformer 104, CFM signal frame data output by the CFM processor 105, and color Doppler image frame data output by the image generator 107. The controller 110 controls each of these components.

Of these, the multiplexer 102, the transmission beamformer 103, the reception beamformer 104, the CFM processor 105, and the image generator 107 constitute an ultrasound signal processing device 150, which includes ultrasound diagnostic processing circuitry.

Elements that constitute the ultrasound diagnostic device 100 and the ultrasound signal processing device 150, for examples, the multiplexer 102, the transmission beamformer 103, the reception beamformer 104, the CFM processor 105, the image generator 107, and the controller 110, may each be implemented by hardware circuits such as field programmable gate arrays (FPGA), application specific integrated circuits (ASIC), or the like.

Circuitry constituting the multiplexer 102, the transmission beamformer 103, the reception beamformer 104, the CFM processor 105, the image generator 107, and the controller 110 may be a computer system comprising a microprocessor and a memory, the memory storing a computer program and the microprocessor operating according to the computer program. For example, the circuitry may include a computer system that operates (or instructs operation of connected elements) according to a computer program of an ultrasound signal processing method of the present invention.

The data storage 109 is a computer-readable storage medium, and may be a flexible disk, hard disk, MO, DVD, DVD-RAM, BD, semiconductor memory, or the like. Further, the data storage 109 may be a storage device that is externally connectable to the ultrasound diagnostic device 100.

The ultrasound diagnostic device 100 pertaining to the present embodiment is not limited to the ultrasound diagnostic device configuration shown in FIG. 1. For example, a configuration without the multiplexer 102 is possible, in which the transmission beamformer 103 and the reception beamformer 104 are directly connected, and connected to the transducers 101a of the probe 101. Further, the transmission beamformer 103 and/or the reception beamformer 104, or a portion thereof, may be integrated into the probe 101. This also applies to the ultrasound diagnostic device pertaining to other embodiments and modifications described later, not only to the ultrasound diagnostic device 100 pertaining to the present embodiment.

### <Description of Elements of Ultrasound Diagnostic Device 100>

### 1. Transmission Beamformer 103

The transmission beamformer 103 is connected to the probe 101 via the multiplexer 102 and controls timing of application of high voltage to each of a plurality of transducers included in a detection wave transmission transducer array Tx (hereinafter, “transducer array Tx”), which corresponds to all or part of the transducers 101a of the probe 101 selected for transmission of a detection wave from the probe 101. For example, assuming the number of the transducers 101a in the probe 101 is 256, all of the transducers 101a may be used as the transducer array Tx. Information indicating position of transducers included in the transducer array Tx is output to the data storage 109 via the control unit 110.

The transmission beamformer 103 includes a transmitter 1031. The transmitter 1031 performs transmission processing to supply a pulsed transmission signal to cause transmission of an ultrasound beam by transducers included in the transducer array Tx, among the transducers 101a of the probe 101, based on a transmission control signal from the controller 110. More specifically, the transmitter 1031 includes, for example, a driver signal generator, a delay profile generator, and a drive signal transmitter. The drive signal generator (not illustrated) is circuitry that generates a transmission pulse signal for causing transmission of an ultrasound beam from transmission transducers corresponding to all or part of the transducers 101a of the probe 101, based on information indicating the transducer array Tx and pulse width from transmission control information from the controller 110. The delay profile generator (not illustrated) is circuitry that outputs settings for each transducer of delay times that determine transmission timing of an ultrasound beam, based on the transducer array Tx from transmission control information from the control 110. The drive signal transmitter (not illustrated) performs detection wave transmission processing supplying a detection wave pulse pwpl (where l is a natural number from 1 to m, referred to as detection wave pulse pwp when it is not necessary to distinguish by number) in order to cause transducers in the transducer array Tx, among the transducers 101a of the probe 101, to transmit an ultrasound beam. The transducer array Tx is selected according to the multiplexer 102. However, configurations pertaining to supply of the detection wave pulse pwp are not limited to the description above. For example, a configuration may be used that does not use the multiplexer 102.

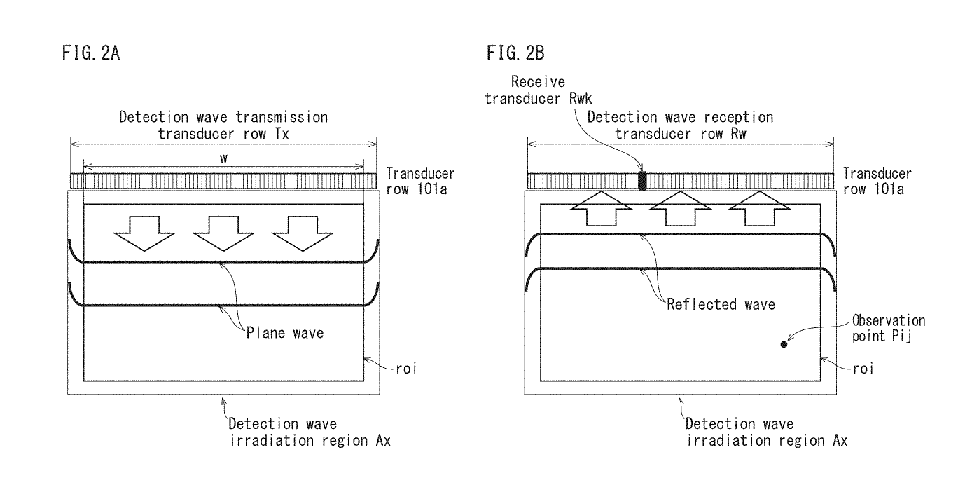

FIG. 2A is a schematic diagram showing configuration of detection wave transmission. A delay time is not applied to transducers included in the transducer array Tx, and detection wave pulses pwp having the same phase are transmitted to the transducer array Tx. Thus, as shown in FIG. 2A, a plane wave that proceeds in a subject depth direction is transmitted from the transducers in the transducer array Tx through at least a region of interest roi that represents an analysis target range in the subject.

Here, a “plane wave” is an unfocused transmission beam having a wavefront shape that does not have a focal point in the subject. A region on a plane including the transducer array Tx and corresponding to a range in the subject to which a detection wave arrives is hereinafter referred to as a detection wave irradiation region Ax. In the detection wave irradiation region Ax, a direction parallel to the transducer array (101a) is referred to as an x direction and a direction perpendicular to the transducer array (101a) is referred to as a z direction.

The transmission beamformer 103 continuously transmits the detection wave pulse pwp a plurality of times, based on the transmission control signal from the controller 110. Continuous detection wave pulse pwp transmission performed from the transducer array Tx is collectively referred to as a “transmission event set”, and each transmission in the transmission event set is referred to as a “transmission event”.

### 2. Reception Beamformer 104

The reception beamformer 104 is a circuit for generating acoustic line signal frame data from electric signals obtained by a plurality of the transducers 101a, based on a reflected wave of a detection wave received by the probe 101. More specifically, the reception beamformer 104 generates an acoustic line signal iₚij for each observation point Pij in a region of interest roi, in order to generate acoustic line signal frame data frame data iₚ(x,z), based on reflected waves from subject tissue received over time by a plurality of the transducers 101a as a result of each of the detection wave pulses pwp. Here, an “acoustic line signal” is a signal from an observation point Pij after reception beamforming processing. Reception beamforming processing is described later.

That is, after transmission of a detection wave pulse pwp, the reception beamformer 104 generates an acoustic line signal iₚij for an observation point Pij from electric signals obtained by a plurality of the transducers 101a based on reflected waves received by the probe 101. Here, i is a natural number from 1 to n, indicating a coordinate in the x direction in the region of interest roi, and j is a natural number from 1 to zₘₐₓ, indicating a coordinate in the z direction. An “acoustic line signal” is a signal focusing a reception signal (RF signal) according to reception beamforming processing.

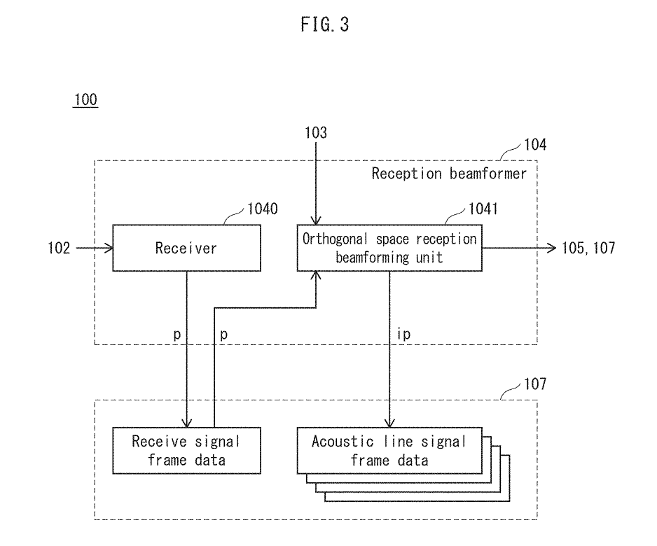

FIG. 3 is function block diagram showing a configuration of the reception beamformer 104. The reception beamformer 104 includes a receiver 1040 and an orthogonal space reception beamforming unit 1041.

The following is a description of each element of the reception beamformer 104.

### 2.1. Receiver 1040

The receiver 1040 is a circuit connected to the probe 101 via the multiplexer 102 that acquires an electric signal for each of a plurality of the transducers 101a, based on reflected waves received over time from the subject, and generates a receive signal plk in a first orthogonal space (t,x) from the azimuth direction x and a time direction t. Here, k is parallel to the azimuth direction x, and is a natural number from 1 to n, indicating a coordinate in the azimuth direction x in the region of interest roi, and l is a natural number from 1 to tₘₐₓ, indicating a coordinate in the time direction t. Further, the receive signal plk is a radio frequency (RF) signal obtained by A/D conversion of an electric signal converted from a reflected wave received by each transducer, based on transmission of a detection wave pulse pwp.

The receiver 1040 generates a sequence in a time direction t of a receive signal plk for each receive transducer rwk for each transmission event, based on reflected waves obtained at each receive transducer rwk (where k is a natural number from 1 to n). Frame data of a receive signal plk is denoted as receive signal frame data p(t,x). Receive signal frame data p(t,x) is obtained from a sequence of receive signals over time t (receive signal sequence) received at a plurality of receive transducers rwk included in a receive transducer array Rw. A receive transducer array Rw (hereinafter, “transducer array Rw”) is composed from part or all of the transducers 101a of the probe 101, and is selected by the multiplexer 102 based on an instruction from the controller 110. The number of transducers in the transducer array Rw may be 32, 64, 96, 128, or 256, for example. According to the present example, all of the transducers 101a are selected as a receive transducer array. Thus, as shown in FIG. 2B, reflected waves from observation points Pij in the entirety of the detection wave irradiation region Ax included in a region of interest roi representing an analysis target range in a subject due to one receive process can be received by using all transducers, in order to generate a receive transducer array of all transducers.

Generated receive signal frame data p(t,x) is outputted to the data storage 109. The data storage 109 inputs receive signal frame data p(t,x) from the receiver 1040 in synchronization with a transmission event, and holds this data until one piece of acoustic line signal frame data iₚ(x,z) is generated from the transmission event.

### 2.2. Orthogonal Space Reception Beamforming Unit 1041

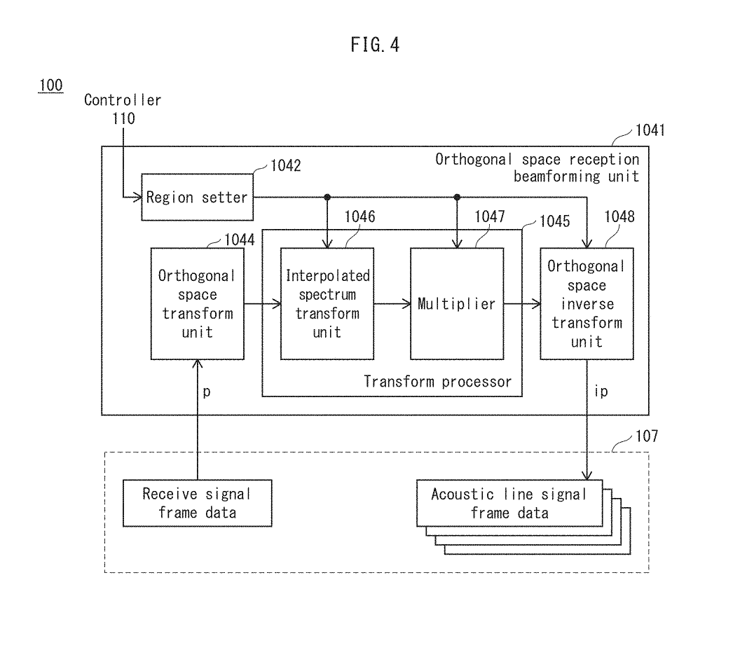

The orthogonal space reception beamforming unit 1041 is circuitry that inputs receive signal frame data p(t,x) generated by the receiver 1040 in synchronization with a transmission event, generates an acoustic line signal iₚij for each observation point Pij in a region of interest roi, and generates acoustic line signal frame data iₚ(x,z). FIG. 4 is a function block diagram showing a configuration of the orthogonal space reception beamforming unit 1041 pertaining to Embodiment 1. As shown in FIG. 4, the orthogonal space reception beamforming unit 1041 includes a region setter 1042, an orthogonal space transform unit 1044, a transform processor 1045 that includes an interpolated spectrum transform unit 1046 and a multiplier 1047, and an orthogonal space inverse transform unit 1048.

The following is a description of each element of the orthogonal space reception beamforming unit 1041.

### (1) Orthogonal Space Transform Unit 1044

The orthogonal space transform unit 1044 is circuitry that converts receive signal frame data p(t,x) in a first orthogonal space (t,x) in a time direction t and an azimuth direction x from the first orthogonal space to a second orthogonal space (ω,κx) that is different from the first orthogonal space, to generate observed spectrum frame data P0. More specifically, the orthogonal space transform unit 1044 performs a two-dimensional Fourier transformation on receive signal frame data p(t,x) in the time direction t and transducer row direction x, to transform to the second orthogonal space (ω,κx) composed of angular frequency ω and transducer row direction x wave number κx, to generate observed spectrum frame data P0. With the exceptions of Embodiments 3 and 4, described later, a fast Fourier transform (FFT) is preferably used in the two-dimensional Fourier transformation. In this case, it is possible to calculate at high speed by setting the number n of transducers included in a receive transducer array Rw to a power of two, such as 32, 64, 96, 128, or 256, for example. Further, in a Fourier transform, weighting processing may be performed by a Hamming window or the like.

Synchronized with a transmission event, generated observed spectrum frame data P0 is outputted to the interpolated spectrum transform unit 1046.

### (2) Region Setter 1042

The region setter 1042 is circuitry that sets a partial region of the observed spectrum frame data P0 as a processing target region in the transform processor 1045. More specifically, the region setter 1042 sets a partial region based on band setting information inputted to the operation input unit 111 by an operator via the controller 110. Band setting information is information indicating a range of angular frequency that indicates frequency in a time direction of the observed spectrum frame data P0. The region setter 1042 sets a rectangular partial region formed by a range of angular frequency ω indicated by band setting information in observed spectrum frame data P0 and an entire range of azimuth direction wavenumber κx.

Information indicating the partial region is outputted to the interpolated spectrum transform unit 1046 and the multiplier 1047 of the transform processor 1045 and the orthogonal space inverse transform unit 1048.

### (3) Transform Processor 1045

The transform processor 1045 is circuitry that inputs observed spectrum frame data P0 and performs predefined calculation processing on observed spectrum partial frame data P0 corresponding to a partial region in a second orthogonal space of the observed spectrum frame data P0 to generate transformed spectrum partial frame data P in a third orthogonal space. The transform processor 1045 includes the interpolated spectrum transform unit 1046 and the multiplier 1047.

The interpolated spectrum transform unit 1046 interpolates angular frequency ω indicating frequency in the time direction in observed spectrum partial frame data P0 with azimuth direction wave number κx and depth direction wave number κz, to generate interpolated spectrum partial frame data P0. The interpolated spectrum partial frame data P0 is outputted to the multiplier 1047.

The multiplier 1047 is circuitry for multiplying interpolated spectrum partial frame data P0 by complex amplitude A to generate transformed spectrum partial frame data P. At this time, the multiplier 1047 inputs information indicating a partial region from the region setter 1042, and multiplies a region of the interpolated spectrum partial frame data P0 corresponding to the partial region by the complex amplitude A. The transformed spectrum partial frame data P is outputted to the orthogonal space inverse transform unit 1048.

### (4) Orthogonal Space Inverse Transform Unit 1048

The orthogonal space inverse transform unit 1048 is circuitry that performs inverse orthogonal transformation on transformed spectrum partial frame data P to an orthogonal space (x,z) of subject depth direction and transducer row direction, to generate acoustic line signals iₚij for observation points Pij in a region of interest, in order to generate acoustic line signal frame data iₚ(x,z). More specifically, the orthogonal space inverse transform unit 1048 performs an inverse Fourier transform on transformed spectrum partial frame data P with the depth direction wavenumber κz and the azimuth direction wavenumber κx to obtain acoustic line signal frame data iₚ(x,z).

For a two-dimensional inverse Fourier transformation, a fast Fourier transform is preferably used. At this time, the orthogonal space inverse transform unit 1048 inputs information indicating a partial region from the region setter 1042, interpolates a frame portion other than the transformed spectrum partial frame data P with dummy data, and then performs an inverse Fourier transform on the entire frame. Acoustic line signal frame data iₚ(x,z) is outputted to and stored by the data storage 109.

### 3. CFM Processor 105

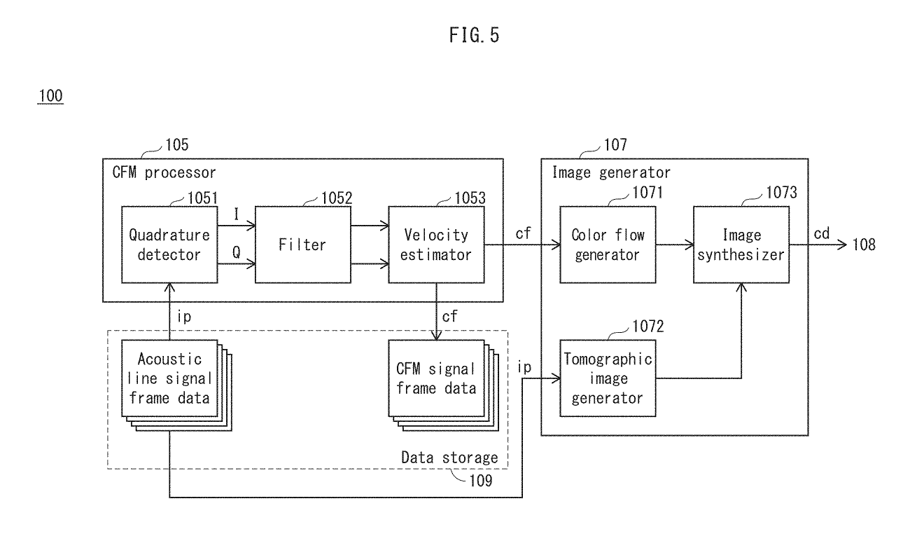

The CFM processor 105 performs frequency analysis based on acoustic line signal frame data iₚ(x,z) obtained in a plurality of transmission event sets, in order to generate CFM signal frame data. Here, “CFM signal” indicates velocity information for an observation point Pij. Velocity information is described later. FIG. 5 is a function block diagram showing a configuration of the CFM processor 105 and the image former 107. As shown in FIG. 5, the CFM processor 105 includes a quadrature detector 1051, a filter 1052, and a velocity estimator 1053.

The following is a description of each element of the CFM processor 105.

### (1) Quadrature Detector 1051

The quadrature detector 1051 is circuitry that performs quadrature detection for each acoustic line signal frame data iₚ(x,z) generated in synchronization with a transmission event and generates a complex acoustic line signal indicating phase of a receive signal at each observation point Pij. More specifically, the following processing is performed. First, a first reference signal having the same frequency as a center frequency of a detection wave and a second reference signal having the same frequency and amplitude as the first reference signal, but different in phase by 90°, are generated. Next, the acoustic line signal and the first reference signal are summed, removing high frequency component having a frequency approximately twice the frequency of the first reference signal by a low-pass filter (LPF), to obtain a first component. Similarly, the acoustic line signal and the second reference signal are summed, removing high frequency component having a frequency approximately twice the frequency of the second reference signal by LPF, to obtain a second component. Finally, a complex acoustic line signal is generated with the first component as a real part (I component; in phase) and the second component as an imaginary part (Q component; quadrature phase).

### (2) Filter 1052

The filter 1052 is filter circuitry that removes clutter from a complex acoustic line signal. Clutter is a component that is not an imaging target among tissue movement, and more specifically is information indicating movement of tissue such as a blood vessel wall, muscle, organ, or the like. Clutter is larger in power than a signal indicating blood flow, but tissue movement is slower than blood flow and therefore has a lower frequency than a signal indicating blood flow. Thus, clutter can be selectively removed. The filter 1052 can apply a known technique such as a wall filter or moving target indicator (MTI).

(3) Velocity estimator 1053

The velocity estimator 1053 is circuitry that estimates movement in the subject that corresponds to each observation point Pij, more specifically blood flow, from complex acoustic line signals after filtering. The velocity estimator 1053 estimates phase for each observation point Pij from complex acoustic line signals corresponding to transmission events pertaining to transmission event sets, and calculates phase shift velocity. At such time, complex acoustic line signals for a given observation point Pij are used without distinction, regardless of which transmission event the complex acoustic line signal was obtained from. The velocity estimator 1053 may estimate phase shift velocity by performing correlation processing between a plurality of complex acoustic line signals.

The velocity estimator 1053 calculates a Doppler shift amount generated at each observation point Pij from phase shift velocity, and estimates average velocity from the Doppler shift amount. The velocity estimator 1053 generates CFM signal frame data in which average velocity is a sequence of signals that are continuous in a transmission direction (depth direction of subject) of a detection wave, and outputs to the image generator 107 and the data storage 109. The velocity estimator 1053 may further calculate dispersion of velocity and/or power, based on a power spectrum of Doppler shift amounts.

### 4. Image Generator 107

The image generator 107 is circuitry that converts acoustic line signal frame data generated by the reception beamformer 104 into a B mode tomographic image, performs color tone conversion on CFM signal frame data generated by the CFM processor 105, and superimposes the color tone converted CFM signal frame data onto the B mode tomographic image. As shown in FIG. 5, the image generator 107 includes a color flow generator 1071, a tomographic image generator 1072, and an image synthesizer 1073.

### (1) Color Flow Generator 1071

The color flow generator 1071 is circuitry that performs color tone conversion for generating a color Doppler image from CFM signal frame data cf. More specifically, initially, the color flow generator 1071 transforms a coordinate system of CFM signal frame data into an orthogonal coordinate system. Next, average velocity of each observation point Pij is transformed to generate color flow data. At such time, for example, a conversion is performed such that (1) a direction towards the probe is red and a direction away from the probe is blue, and (2) saturation is higher the larger the absolute value of velocity and saturation is lower the smaller the absolute value of velocity. More specifically, for a velocity component towards the probe, absolute value of velocity is converted to a red luminance value, and for a velocity component away from the probe, absolute value of velocity is converted to a blue luminance value.

The color flow generator 1071 may further receive a signal indicating velocity dispersion from the CFM processor 105, and convert the dispersion value into a green luminance value. In this way, it is possible to show where turbulence occurs.

The color flow generator 1071 outputs generated color flow information to the image synthesizer 1073.

### (2) Tomographic Image Generator 1072

The tomographic image generator 1072 is circuitry that generates a B mode tomographic image from acoustic line signal frame data iₚ. More specifically, initially, the tomographic image generator 1072 transforms a coordinate system of acoustic line signal frame data iₚ into an orthogonal coordinate system. Next, the tomographic image generator converts values of acoustic line signals for each observation point Pij into luminance to generate a B mode tomographic image. More specifically, the tomographic image generator 1072 performs envelope detection on values of acoustic line signals, and converts the values to luminance by performing logarithmic compression. The tomographic image generator 1072 outputs a generated B mode tomographic image to the image synthesizer 1073.

### (3) Image Synthesizer 1073

The image synthesizer 1073 is circuitry that superimposes color flow information generated by the color flow generator 1071 on a B mode tomographic image generated by the tomographic image generator 1072, generating a color Doppler image cd, and outputs to the display 108. Thus, a color Doppler image cd in which direction and speed (absolute value of velocity) of blood flow are added on a B mode tomographic image is displayed on the display 108.

### <Operations>

The following describes operations of the ultrasound diagnostic device 100 configured as described above.

### 1. Overview of Operations of Ultrasound Diagnostic Device 100

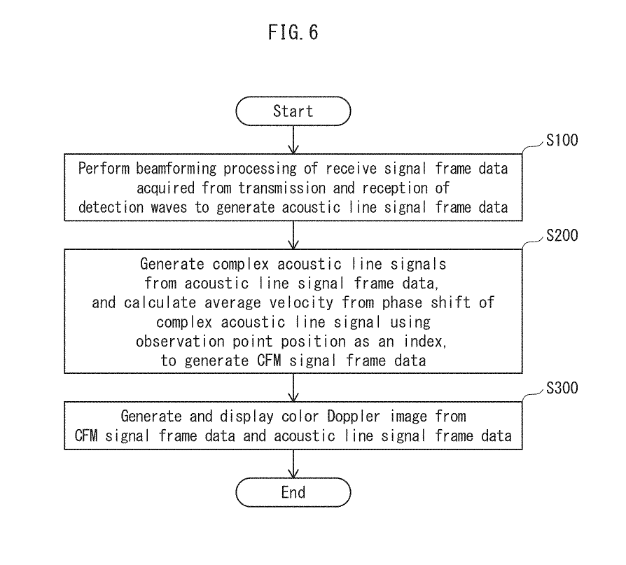

FIG. 6 is a flowchart showing operations of the ultrasound diagnostic device 100.

First, in step S100, reception beamforming processing is performed on receive signal frame data p(t,x) acquired through transmission and reception of a detection wave, in order to generate acoustic line signal frame data iₚ(x,z). Here, transmission processing and reception processing are performed once for each target region (that is, a transmission event set including only one transmission event), and each transducer receives a receive signal series (RF signal) based on a detection wave in a time series from a subject, in order to generate receive signal frame data p(t,x), and perform reception beamforming processing to generate acoustic line signal frame data iₚ(x,z). Generated acoustic line signal frame data iₚ(x,z) is outputted to the image generator 107 and the data storage 109. Details of the reception beamforming processing in step S100 are provided later.

Next, in step S200, the CFM processor 105 reads acoustic line signal frame data iₚ(x,z) stored in the data storage 109, and calculates average velocity phase shift of complex acoustic line signals, with positions of observation points Pij as an index. Initially, the quadrature detector 1051 performs quadrature detection for each acoustic line signal read, transforming to complex acoustic line signals. The filter 1052 removes or reduces clutter for each complex acoustic line signal. Next, the velocity estimator 1053 estimates phase shift velocity by performing correlation processing on a plurality of complex acoustic line signals pertaining to the same observation point Pij. At such time, as described above, as long as the complex acoustic line signals pertain to the same observation point Pij, no distinction is made between acoustic line signals pertaining to different transmission event sets. Further, the velocity estimator 1053 calculates Doppler shift amounts from estimated phase shift velocity, calculates velocity from Doppler shift amounts, and calculates average values of velocity. The velocity estimator 1053 may calculate average velocity based on an average value of Doppler shift amounts, and may calculate average Doppler shift amounts from average values of estimated phase shift velocity. Finally, the velocity estimator 1053 associates calculated average velocity with observation points Pij, generates CFM signal frame data cf(x,z), and outputs to the image generator 107 and the data storage 109.

Next, in step S300, the image generator 107 generates and displays a color Doppler image. The color flow generator 1071 generates color flow information from CFM signal frame data cf(x,z), and the tomographic image generator 1072 generates B mode tomographic image from acoustic line signal frame data iₚ(x,z). Finally, the image synthesizer 1073 superimposes color flow information on a B mode tomographic image to generate a color Doppler image cd(x,z) and outputs to the display 108.

### 2. Reception Beamforming Processing Operation in Step S100

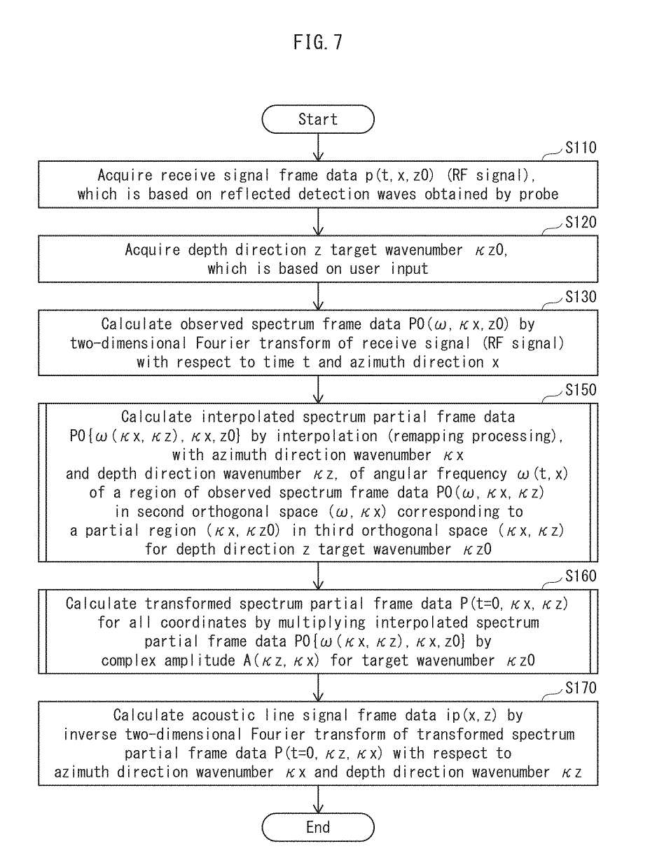

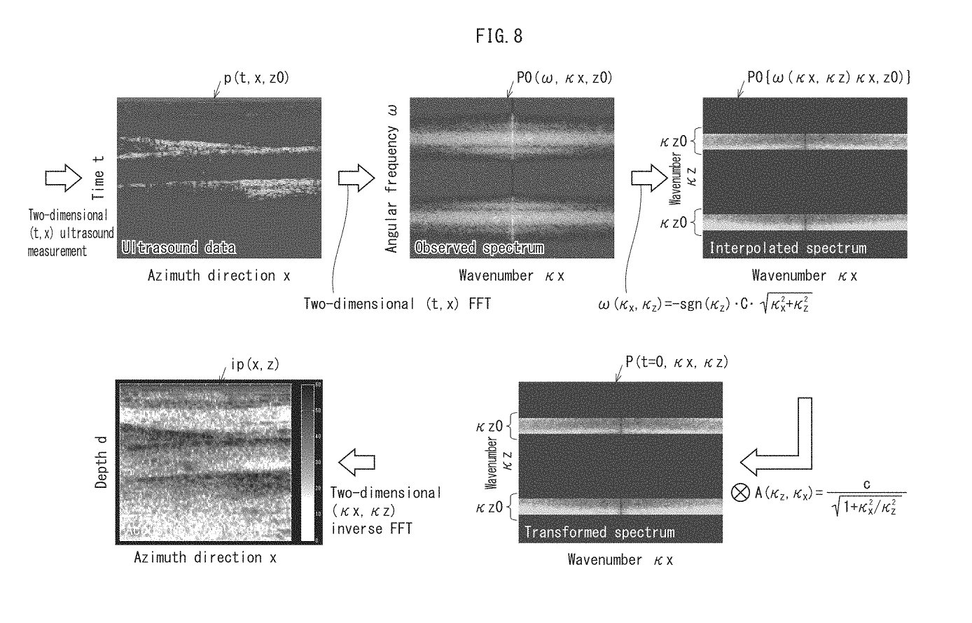

Details of the reception beamforming processing in step S100 are provided below. FIG. 7 is a flowchart showing an overview of reception beamforming operations. FIG. 8 is a schematic diagram showing aspects of frame data or partial frame data (hereinafter, “map”) obtained by reception beamforming.

Initially, in step S110, the orthogonal space transform unit 1044 acquires receive signal frame data p(t,x,z0) generated by the receiver 1040 based on reflected detection waves obtained by the probe 101. The top left side of FIG. 8 shows a schematic diagram of receive signal frame data p(t,x,z0). In the map at the top left side of FIG. 8, the vertical axis represents time, and the horizontal axis represents azimuth direction x. “z0” represents a position in a subject depth direction z, in which a surface of the subject is z0 (z=0).

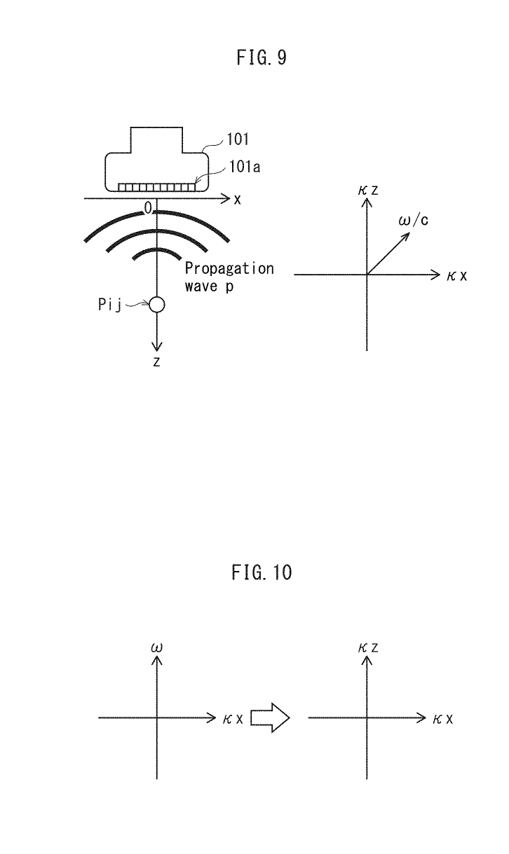

FIG. 9 is a schematic diagram showing a relationship between a propagation wave p(t,x,z) and a wave vector in the subject. The relationship between propagation wave p(t,x,z) and wave vector can be represented by Expression (1), using ω, κx, κz (ω: time direction t angular frequency, κx: azimuth direction x wavenumber, κz: subject depth direction z wavenumber).

[Expression 1]

p(t,x,z)∝e^{i(k}^{ₓ}^{x+k}^{_{z}}^{z-ωt)} (1)

As shown on FIG. 9, a receive signal acquired by the probe 101 indicates value of a propagation wave p(t,x,z) at the subject surface z0 where the probe 101 is positioned, and therefore receive signal frame data acquired at the subject surface z0 can be expressed as p(t,x,z0).

Next, in step S120, the region setter 1042 acquires the depth direction z target wavenumber κz0 to be processed, based on operator input inputted to the operation input unit 111. Thus, the operator determines a condition of target wavenumber κz0 for remapping.

In step S130, the orthogonal space transform unit 1044 performs a two-dimensional Fourier transform in time t and azimuth direction x on receive signal frame data p(t,x,z0) in the first orthogonal space (t,x) composed of the time direction t and azimuth direction x, and transforms observed spectrum frame data P0(ω, κx, z0) in the second orthogonal space (ω, κx) composed of angular frequency ω and azimuth direction wavenumber κx. For Fourier transformation, a fast Fourier transform is preferably used.

Receive signal frame data p(t,x,z0) and observed spectrum frame data P0(ω,κx,z0) can be expressed by Expression (2) according to a relationship of two-dimensional Fourier transformation.

$$\begin{matrix}[ Expression 2 ] & \\ {P}_{0} ( ω , {κ}_{x} , {z}_{0} ) = \frac{1}{4 {π}^{2}} {\int \int }_{- ∞}^{∞} p ( t , x , {z}_{0} ) {e}^{- {ik}_{x} x} {e}^{i ω t} dxdt & ( 2 )\end{matrix}$$

The top center of FIG. 8 shows a schematic diagram of observed spectrum frame data P0(ω,κx,z0). In the map at the top center of FIG. 8, the vertical axis represents angular frequency, the horizontal axis represents azimuth direction x wavenumber κx, and a center of the map (ω,κx)=(0,0) indicates an origin point (0,0). In the vertical axis, angular frequency is observed as distribution centered on detection wave transmission frequency.

In subsequent steps S150-S160, observed spectrum frame data P0(ω,κx,z0) in second orthogonal space (ω,κx) is transformed to transformed spectrum partial frame data P(t=0,κx,κz) in third orthogonal space (κx,κz).

Initially, in step S150, the interpolated spectrum transform unit 1046 acquires depth direction z target wavenumber κz0 from the region setter 1042, performs remapping processing on angular frequency ω(t,x) of a range of observed spectrum frame data P0(ω,κx,z0) in second orthogonal space (ω,κx) corresponding to a partial region (κx,κz0) in third orthogonal space (κx,κz), interpolating with azimuth direction wavenumber κx and depth direction wavenumber κz to calculate interpolated spectrum partial frame data P0{ω(κx,κz),κx,z0}.

Here, when sound velocity is c, a relationship between wavenumber κx, wavenumber κz, and angular frequency ω is shown by Expression (3).

$$\begin{matrix}[ Expression 3 ] & \\ \frac{{ω}^{2}}{{c}^{2}} = {κ}_{x}^{2} + {κ}_{z}^{2} & ( 3 )\end{matrix}$$

Here, ω(κx,κz) in interpolated spectrum partial frame data P0{ω(κx,κz),κx,z0} is expressed by Expression (4). According to Expression (4), observed spectrum partial frame data P0(ω,κx,z0) can be transformed to interpolated spectrum partial frame data P0{ω(κx,κz),κx,z0} by interpolating depth direction wavenumber κz with target wavenumber κz0 and interpolating azimuth direction wavenumber κx with an entire wavenumber range.

[Expression 4]

ω(Kₓ,K_{z})=−sgn(K_{z})·c·\sqrt{Kₓ²+K_{z}²} (4)

The top right side of FIG. 8 shows a schematic diagram of interpolated spectrum partial frame data P0{ω(κx,κz),κx,z0}. In the map at the top right of FIG. 8, the vertical axis represents depth direction z wavenumber κz, the horizontal axis represents azimuth direction x wavenumber κx, and a center of the map (κx,κz)=(0,0) indicates an origin point (0,0). In the vertical axis, wavenumber κz is observed as distribution centered on detection wave transmission frequency. It can be seen that interpolated spectrum data P0 is calculated for depth direction wavenumber κz in a limited range included in target wavenumber κz0, and for azimuth direction wavenumber κx included in an entire wavenumber range.

In step S160, for a range of target wave number κz0 of wave number κz in the depth direction and a range of wave number κx included in an entire wavenumber range in the third orthogonal space (κx,κz), interpolated spectrum partial frame data P0{ω(κx,κz)κx,z0} is multiplied by complex amplitude A(κz,κx) to calculate transformed spectrum partial frame data P(t=0,κx,κz) by using Expression (5).

[Expression 5]

P(t=0,Kₓ,K_{z})=A(K_{z},Kₓ)·P₀{ω(Kₓ,K_{z}),Kₓ,Z₀} (5)

Here, complex amplitude A (κz,κx) is represented by Expression (6).

$$\begin{matrix}[ Expression 6 ] & \\ A ( {κ}_{z} , {κ}_{x} ) = \frac{\partial ω ( {κ}_{z} , {κ}_{x} )}{\partial {κ}_{z}} = \frac{c}{\sqrt{1 + {κ}_{x}^{2} / {κ}_{z}^{2}}} & ( 6 )\end{matrix}$$

According to the calculations above, a transformation from the second orthogonal space (ω,κx) to the third orthogonal space (κx,κz), as shown in FIG. 10, is performed.

The bottom right side of FIG. 8 shows a schematic diagram of transformed spectrum partial frame data P(t=0,κx,κz). In the map at the bottom right of FIG. 8, the vertical axis represents subject depth direction z wavenumber κz, the horizontal axis represents azimuth direction x wavenumber κx, and a center of the map (κx,κz)=(0,0) indicates an origin point (0,0). It can be seen that transformed spectrum data P is calculated for depth direction wavenumber κz in a limited range included in target wavenumber κz0, and for azimuth direction wavenumber κx included in an entire wavenumber range.

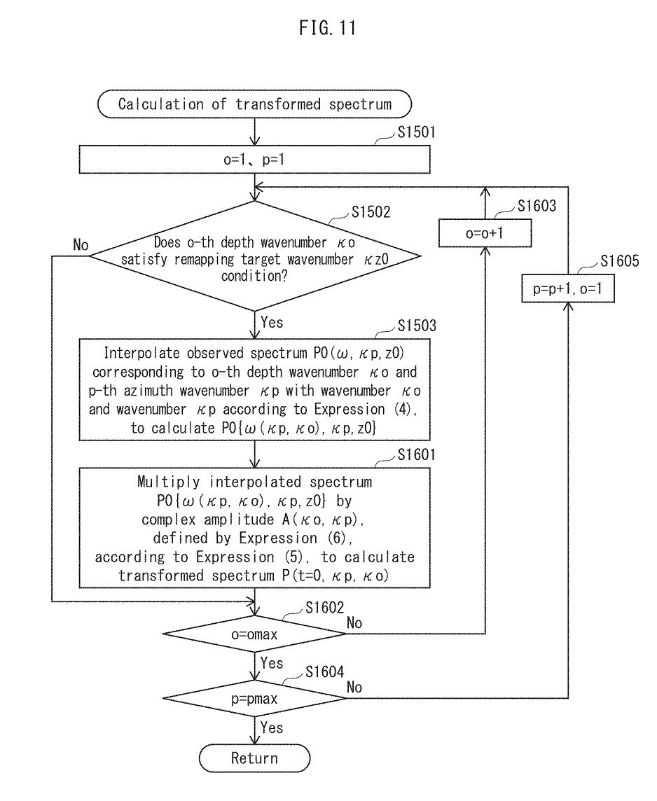

The following describes details of calculation of interpolated spectrum in steps S150 and S160. FIG. 11 is a flowchart showing details of operations in steps S150 and S160.

Initially, o and p are initialized (step S1501), and whether or not o-th depth wavenumber κo satisfies a mapping target wavenumber κz0 condition is determined (step S1502). When the condition is not satisfied, processing proceeds to step S1602, and when the condition is satisfied, processing proceeds to step S1503.

In step S1503, interpolated spectrum P0{ω(κp,κo),κp,z0} is calculated by interpolating ω in observed spectrum P0(ω,κp,z0), corresponding to p-th azimuth wavenumber κp, with wavenumber κo and wavenumber κp, according to Expression (4). In step S1504, complex amplitude A(κo,κp) defined by Expression (6) is multiplied by interpolated spectrum P0{ω(κp,κo),κp,z0} according to Expression (5) to calculate transformed spectrum data P(t=0,κp,κo), and processing proceeds to step S1602. If o is not a maximum value omax of wavenumber κo, o is incremented and processing returns to step S1502; if o is the maximum value omax, processing proceeds to step S1604. Further, if p is not a maximum value pmax of wavenumber κp, p is incremented and processing returns to step S1502; if p is the maximum value pmax, interpolated spectrum calculation processing ends.

Returning to FIG. 7, according to the subsequent step S170, calculation of acoustic line signal frame data iₚ(x,z) is performed.

In step S170, the transformed spectrum partial frame data P(t=0,κx,κz) is further transformed by a two-dimensional inverse Fourier transform on azimuth direction wavenumber κx and depth direction wavenumber κz to calculate acoustic line signal frame data iₚ(x,z) in orthogonal space (x,z). As described above, in steps S150-S160, observed spectrum frame data P0(ω,κx,z0) in second orthogonal space (ω,κx) is transformed to transformed spectrum partial frame data P(t=0,κx,κz) in third orthogonal space (κx,κz) according to Expressions (4) to (6). Using transformed spectrum partial frame data P(t=0,κx,κz) in third orthogonal space (κx,κz), acoustic line signal frame data iₚ(x,z) can be calculated by using Expression (7). Here, Δz represents a difference in depth between subject depth direction coordinate z and subject surface z0.

$$\begin{matrix}[ Expression 7 ] & \\ {i}_{p} ( x , {z}_{0} - Δ z ) = {\int \int }_{- ∞}^{∞} P ( t = 0 , {κ}_{x} , {κ}_{z} ) \cdot {e}^{i {κ}_{z} Δ z} \cdot {e}^{{ik}_{x}} d {κ}_{x} d {κ}_{z} & ( 7 )\end{matrix}$$

When acoustic line signal frame data iₚ(x,z) is calculated, the reception beamforming processing operations in step S100 end.

The bottom left side of FIG. 8 shows a schematic diagram of acoustic line signal frame data iₚ(x,z). In the map at the bottom left side of FIG. 8, the vertical axis represents subject depth direction z, and the horizontal axis represents azimuth direction x. As described above, in the transformed spectrum partial frame data P(t=0,κx,κz), in a limited range included in target wavenumber κz0 of depth direction wavenumber κz, a range of azimuth direction wavenumber κx included in an entire wavenumber range is calculated. In contrast, focused acoustic line signal frame data iₚ(x,z) can be obtained for an entire region of subject depth direction z and azimuth direction x in orthogonal space (x,z) by a two-dimensional inverse Fourier transform on azimuth direction wavenumber κx and depth direction wavenumber κz of transformed spectrum partial frame data P in the second orthogonal space. That is, it is possible to improve frame rate while reducing the amount of calculation in reception beamforming while suppressing deterioration of quality of an ultrasound image.

### <Summary>

1. The ultrasound diagnostic device 100 pertaining to the embodiment described above includes: a receiver 1040 that acquires, for each transducer 101a, a receive signal sequence based on reflected detection waves received in a time sequence from the subject, to generate receive signal frame data p(t,x,z0) in a first orthogonal space (t,x) defined by a time direction t and a transducer row direction x; an orthogonal space transform unit 1044 that transforms the receive signal frame data p(t,x,z0) from the first orthogonal space (t,x) to a second orthogonal space (ω,κx), to generate observed spectrum frame data P0(ω,κx,z0); a transform processor 1045 that performs predefined calculation processing on observed spectrum partial frame data P corresponding to a partial region in the second orthogonal space (ω,κx) of the observed spectrum frame data P0(ω,κx,z0), to generate transformed spectrum partial frame data P(t=0,κx,κz) in a third orthogonal space (κx,κz); and an orthogonal space inverse transform unit 1048 that performs an inverse orthogonal transform on the transformed spectrum partial frame data P(y=0,κx,κz) to an orthogonal space (x,z) defined by a subject depth direction z and the transducer row direction x, to generate acoustic line signals for the observation points Pij in the region of interest roi, in order to generate the acoustic line signal frame data iₚ(x,z).

According to this configuration, it is possible to reduce the amount of calculation in reception beamforming of a reflected wave obtained from ultrasound transmission to a subject. As a result, frame rate can be improved while suppressing deterioration in image quality.

According to another configuration, the orthogonal space transform unit 1044 transforms the receive signal frame data p(t,x,z0) into the second orthogonal space (ω,κx) by performing a Fourier transform with respect to the time direction t and the transducer row direction x to generate the observed spectrum frame data P0(ω,κx,z0), and the orthogonal space inverse transform unit 1048 transforms the transformed spectrum partial frame data P(t=0,κx,κz) by performing an inverse Fourier transform with respect to subject depth direction wavenumber κz and transducer row direction wavenumber κx to generate the acoustic line signal frame data iₚ(x,z).

According to this configuration, the receive signal frame data p(t,x,z0) in the first orthogonal space (t,x) can be transformed into the second orthogonal space (ω,κx)to generate the observed spectrum frame data P0(ω,κx,z0), and the transformed spectrum partial frame data P(t=0,κx,κz) in the third orthogonal space (κx,κz) can be transformed into the orthogonal space (x,t) to generate the acoustic line signal frame data.

According to another configuration, the transform processor 1045 includes: an interpolated spectrum transform unit 1046 that interpolates time direction angular frequency ω in the observed spectrum partial frame data P0(ω,κx,z0) with transducer row direction wavenumber κx and subject depth direction wavenumber κz, to generate interpolated spectrum partial frame data P0{ω(κx,κz),κx,z0}; and a multiplier 1047 that multiplies the interpolated spectrum partial frame data P0{ω(κx,κz),κx,z0} by a complex amplitude to generate the transformed spectrum partial frame data P(t=0,κx,κz).

According to this configuration, observed spectrum partial frame data P0(ω,κx,z0) in the second orthogonal space (ω,κx) can be transformed to transformed spectrum partial frame data P(t=0,κx,κz) in the third orthogonal space 3, and a calculation amount for reception beamforming can be reduced.

### 2. Use of Plane Wave

According to the aspect shown by the embodiment, a plane wave that proceeds in a subject depth direction is transmitted from the transducers in the transducer array Tx through at least a region of interest roi that represents an analysis target range in the subject. Thus, reflected waves from observation points Pij in the entirety of the detection wave irradiation region Ax included in a region of interest roi representing an analysis target range in a subject due to one transmission and reception process can be received by using all transducers, in order to generate a receive transducer array of all transducers. As above, transmission and reception of a plane wave is a preferable method for improving frame rate. Accordingly, in the ultrasound signal processing method of the present disclosure, the transmission beamformer preferably transmits an unfocused ultrasound beam that does not have a focal point in a subject. By transmitting a plane wave as a detection wave, it is possible to further improve frame rate while reducing the amount of calculation in reception beamforming while suppressing deterioration of quality of an ultrasound image.

### 3. Use of CFM Method

According to the aspect shown in the embodiment above, the image generator preferably has a configuration in which acoustic line signal image frame data is based on phase information of acoustic line signal frame data. According to a CFM method, a Doppler shift (frequency deviation) occurring in an echo due to movement of body tissue such as blood flow is detected from a phase difference between a transmitted wave and a reflected wave, and speed information in the form of a two-dimensional image is superimposed on a two-dimensional image (B mode tomographic image). In order to detect Doppler shift, transmission and reception of ultrasound is repeated while transmission frequency is limited to a specific frequency at the same position in a subject, and phase differences between transmitted waves and reflected waves are measured. Accordingly, the reception beamforming of the present disclosure, including a region setter that sets a partial region of observed spectrum frame data as a processing target region according to a range of time direction angular frequency and all of a transducer row direction wavenumber, is similar to frequency analysis by the CFM processor 105, and output of acoustic line signal by the reception beamformer of the present disclosure can preferably be used for generating CFM signal frame data by the CFM processor. In addition, as processing after reception beamforming based on phase differences between transmission waves and reflected waves, a pulse Doppler method, continuous wave Doppler method, tissue Doppler method, strain elasticity method, shear wave elasticity method, and the like are suitable as applications of reception beamformer output of the present disclosure. Application of acoustic line signal output of the reception beamformer of the present disclosure to CFM signal, when compared to application to a B mode tomographic image, has less ultrasound image quality deterioration while limiting angular frequency of observed spectrum frame data.

### Embodiment 2

According to the ultrasound diagnostic device 100 pertaining to Embodiment 1, the region setter 1042 sets a partial region of observed spectrum frame data P0, based on band setting information inputted to the operation input unit 111 by an operator and acquired via the controller 110, to set a processing target region for the transform processor 1045.

However, a method of setting a processing target region is not limited to the above, and may be appropriately modified to be set by observed spectrum frame data P0.

The following describes an ultrasound diagnostic device 100A pertaining to Embodiment 2.

### <Configuration>

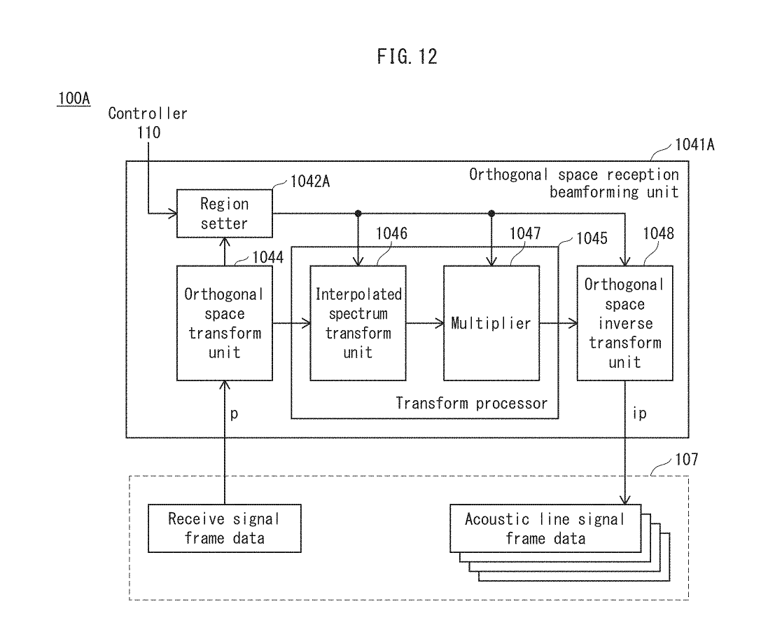

According to the ultrasound diagnostic device 100A pertaining to Embodiment 2, an orthogonal space reception beamforming unit 1041A is different from its equivalent in the ultrasound diagnostic device 100 pertaining to Embodiment 1, and this configuration is described below. Other configuration is the same as for the ultrasound diagnostic device 100 and description thereof is omitted. FIG. 12 is a function block diagram showing a configuration of the orthogonal space reception beamforming unit 1041A pertaining to Embodiment 2. Configuration of the region setter 1042A is different from its equivalent in Embodiment 1, and therefore described below. Other configuration is the same as for the ultrasound diagnostic device 100 and description thereof is omitted.

The region setter 1042A is circuitry that sets a partial region of observed spectrum frame data P0 as a processing target region for the transform processor 1045, based on the observed spectrum frame data P0. More specifically, the region setter 1042A sets a partial region as a processing target region, based on observed spectrum frame data P0 inputted from the orthogonal transform unit 1044. Band setting information is information indicating a range of angular frequency ω that indicates frequency in a time direction of the observed spectrum frame data P0. The region setter 1042A sets a partial region formed by a range of angular frequency ω indicated by band setting information in observed spectrum frame data P0 and an entire range of azimuth direction x wavenumber κx.

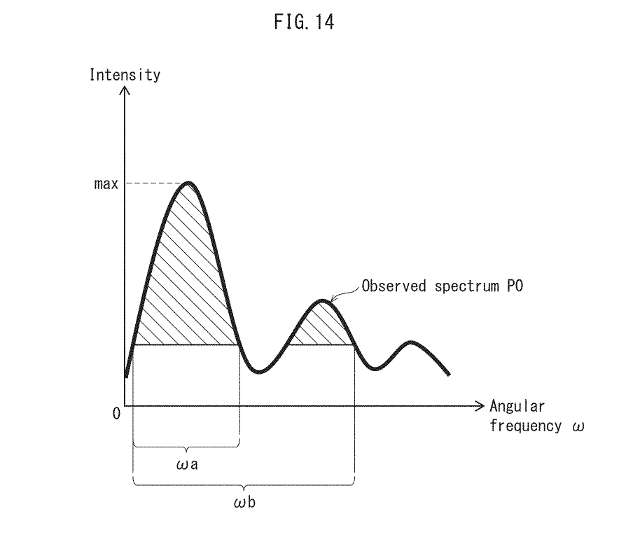

FIG. 14 is an explanatory diagram showing partial regions of observed spectrum frame data P0, which are processing target regions in reception beamforming pertaining to Embodiment 2. The curved line in the figure is a graph showing distribution of intensity of observed spectrum frame data P0 at angular frequency ω.

As shown in FIG. 14, the region setter 1042A sets a partial region to be a processing target region, based on a frequency band ωa that includes a maximum intensity of the observed spectrum frame data P0. At this time, the region setter 1042A may be configured to set a partial region to be a processing target region for each transducer row direction x wavenumber κx, based on the frequency band ωa that includes a maximum intensity of the observed spectrum frame data P0.

Alternatively, the region setter 1042A may set a partial region to be a processing target region based on a frequency band ωb that exceeds the frequency band ωa that includes a maximum intensity of the observed spectrum frame data P0. In this case also, the region setter 1042A may be configured to set a partial region to be a processing target region for each transducer row direction x wavenumber κx, based on the frequency band ωb that is determined based on frequencies at which maximum intensities are obtained for the observed spectrum frame data P0.

The region setter 1042A may set a partial region to be a processing target region based on an observed spectrum frame data P0 obtained from each transmission event, but, for example, may alternatively set the partial region for a predefined number of detection wave transmissions.

Information indicating the partial region is outputted to the interpolated spectrum transform unit 1046 and the multiplier 1047 of the transform processor 1045 and the orthogonal space inverse transform unit 1048.

### <Operations>

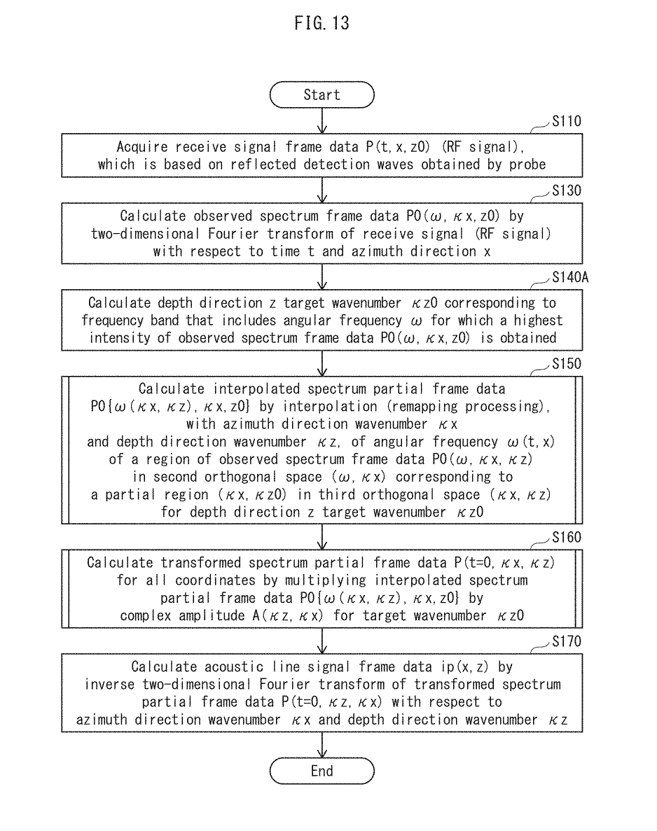

The following describes operations of the ultrasound diagnostic device 100A. FIG. 13 is a flowchart showing an overview of reception beamforming operations of the ultrasound diagnostic device 100A pertaining to Embodiment 2. Operations of the ultrasound diagnostic device 100A are the same as the processing shown in FIG. 6 and FIG. 7, with the exception of step S140A in FIG. 13, and therefore only different processing is described.

In step S140A in FIG. 13, the region setter 1042A sets a partial region of observed spectrum frame data P0(ω,κx,z0) to be a processing target region in steps S150 and S160, based on a frequency band ωa that includes a maximum obtained angular frequency ω in the observed spectrum frame data P0(ω,κx,z0). A partial region of observed spectrum frame data P0(ω,κx,z0) is a region of frame data composed from a limited angular frequency ω and an entire range of azimuth direction wavenumber κx in observed spectrum frame data P0(ω,κx,z0) in the second orthogonal space (ω,κx).

In steps S150 and S160, with respect to a partial region in third orthogonal space (κx,κz) corresponding to the partial region of observed spectrum frame data P0(ω,κx,z0), observed spectrum frame data P0(ω,κx,z0) in second orthogonal space (ω,κx) is transformed to transformed spectrum partial frame data P(t=0,κx,κz) in third orthogonal space (κx,κz). A partial region in third orthogonal space (κx,κz) corresponding to a partial region of observed spectrum frame data P0(t=0,ω,κx) is a region of frame data composed from a limited range of subject depth direction z wave number κz and an entire range of azimuth direction x wavenumber κx in transformed spectrum partial frame data P(t=0,κx,κz) in third orthogonal space (κx,κz).

As described above, according to Embodiment 2, a partial region to be a processing target region can be set based on a predefined frequency band ωa that includes a maximum intensity of observed spectrum frame data P0(ω,κx,z0), which is orthogonally transformed from receive signal frame data p(t,x,z0), and therefore observed spectrum frame data P0(ω,κx,z0) can be obtained based on a signal of frequency at which a maximum intensity of a reflected detected wave is obtained. Thus, it is possible to improve frame rate while reducing the amount of calculation in reception beamforming while further suppressing deterioration of quality of an ultrasound image.

### <Summary>

As described above, according to Embodiment 2, a partial region of observed spectrum frame data P0(ω,κx,z0) is composed from a range of time direction t angular frequency ω and an entire range of transducer row direction x wavenumber κx, the receive beamformer 104 includes the region setter 1042A that sets the partial region of the observed spectrum frame data P0(ω,κx,z0) as a processing target region, the region setter 1042A being configured to set a range of frequency band ωa that includes a frequency of maximum intensity in the observed spectrum frame data P0(ω,κx,z0). The region setter 1042A may be configured to set a range of frequency band ωa that includes a frequency of maximum intensity in the observed spectrum frame data P0(ω,κx,z0) for each transducer row direction x wavenumber κx.

Further, the region setter 1042A may be configured to set frequency range ωb that includes a frequency of maximum intensity in observed spectrum frame data P0(ω,κx,z0). In this case, the region setter 1042A may be configured to set a range of frequency range ωb that includes a frequency of maximum intensity in the observed spectrum frame data P0(ω,κx,z0) for each transducer row direction x wavenumber κx.

According to this configuration, a partial region to be a processing target region can be set based on a predefined frequency band ωa or frequency range ωb that includes a maximum intensity of observed spectrum frame data P0(ω,κx,z0), which is orthogonally transformed from receive signal frame data p(t,x,z0), and therefore observed spectrum frame data P0(ω,κx,z0) can be obtained based on a signal of a frequency at which a maximum intensity of a reflected detected wave is obtained. As a result, an amount of calculation in reception beamforming of a reflected wave obtained from ultrasound transmission into a subject can be reduced, while suppressing deterioration in ultrasound image quality. Thus, it is possible to improve frame rate while further suppressing deterioration of ultrasound image quality.

### Embodiment 3

According to the ultrasound diagnostic device 100A pertaining to Embodiment 2, the orthogonal space inverse transform unit 1048 generates acoustic line signal frame data by an inverse Fourier transform using a fast Fourier transform on transformed spectrum partial frame data in subject depth direction wavenumber κz and azimuth direction wavenumber κx.

However, processing methods for inverse Fourier transform are not limited to the above, and inverse Fourier transform may be applied to a limited target wavenumber κz0 of the subject depth direction wavenumber κz.

The following describes an ultrasound diagnostic device 100B pertaining to Embodiment 3.

### <Configuration>

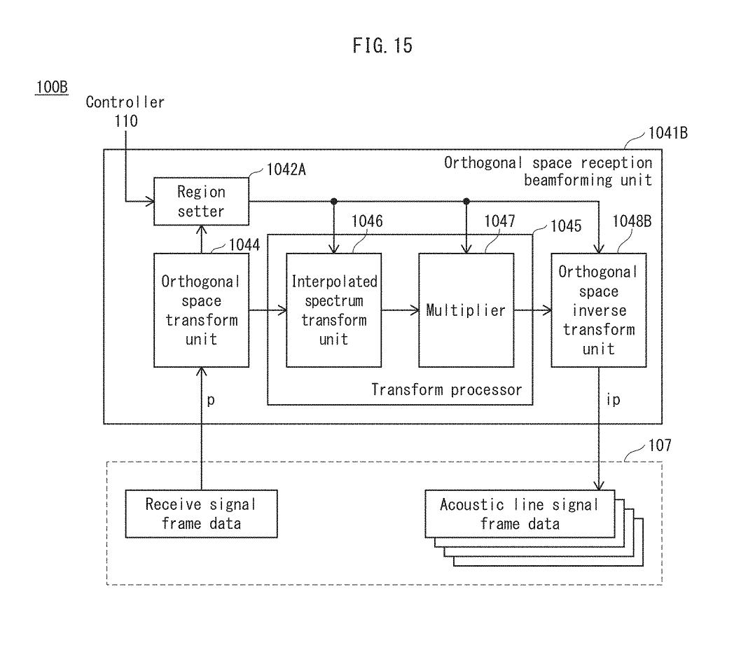

According to the ultrasound diagnostic device 100B pertaining to Embodiment 3, an orthogonal space reception beamforming unit 1041B is different from its equivalent in the ultrasound diagnostic device 100A pertaining to Embodiment 2, and this configuration is described below. Other configuration is the same as for the ultrasound diagnostic device 100A and description thereof is omitted. FIG. 15 is a function block diagram showing a configuration of the orthogonal space reception beamforming unit 1041B pertaining to Embodiment 3. Configuration of an orthogonal space inverse transform unit 104B is different from its equivalent in Embodiment 2, and therefore described below. Other configuration is the same as for the ultrasound diagnostic device 100A and description thereof is omitted.

The orthogonal space inverse transform unit 1048B performs inverse Fourier transform by using a discrete Fourier transform (DFT) on transformed spectrum partial frame data with respect to a limited target wavenumber κz0 of subject depth direction wavenumber κz. On the other hand, acoustic line signal frame data is generated by performing inverse Fourier transform by using a fast Fourier transform with respect to all of wavenumber κx with respect to azimuth direction wavenumber κx. At this time, the orthogonal space inverse transform unit 1048B inputs information indicating a range from the region setter 1042A, interpolates a frame portion other than the transformed spectrum partial frame data with dummy data, and then performs an inverse Fourier transform on the entire frame. Acoustic line signal frame data is outputted to and stored by the data storage 109.

### <Operations>

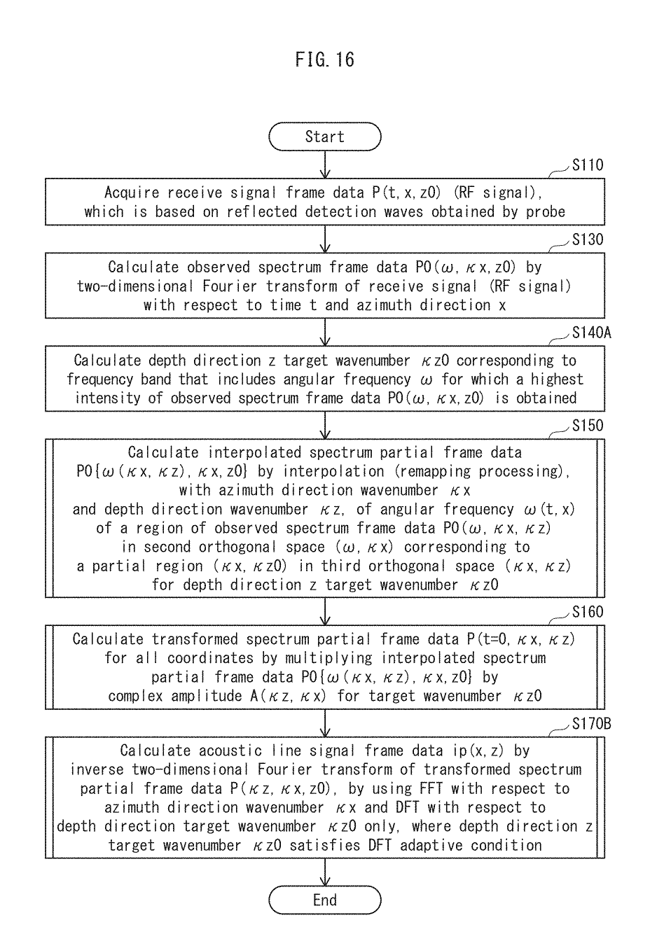

The following describes operations of the ultrasound diagnostic device 100B. FIG. 16 is a flowchart showing an overview of reception beamforming operations of the ultrasound diagnostic device 100B. Operations of the ultrasound diagnostic device 100B are the same as the processing shown in FIG. 6 and FIG. 14, with the exception of step S170B in FIG. 16, and therefore only different processing is described.

In step S170B in FIG. 16, the orthogonal space inverse transform unit 1048B, when target wavenumber κz0 satisfies an adaptive condition of an inverse discrete Fourier transformation, inverse Fourier transform of transformed spectrum partial frame data P(t=0,κx,κz) is performed by using inverse discrete Fourier transform with respect to limited target wavenumber κz0 of subject depth direction wavenumber κz. On the other hand, acoustic line signal frame data iₚ(x,z) in orthogonal space (t,x) is calculated by performing inverse Fourier transform by using a fast Fourier transform with respect to all of wavenumber κx of azimuth direction wavenumber κx.

The orthogonal space inverse transform unit 1048B determines whether or not target wavenumber κz0 satisfies an adaptive condition of inverse discrete Fourier transform according to Expressions (8) and (9), where n is a data number, m is a Fourier transform data number, fw is a Fourier transform frequency band, and fs is a sampling frequency scheme.

$$\begin{matrix}[ Expression 8 ] & \\ m \leq \sqrt{n {log}_{2} n} & ( 8 ) \\ [ Expression 9 ] & \\ m = \frac{{f}_{w}}{{f}_{s}} n & ( 9 )\end{matrix}$$

Using transformed spectrum partial frame data P(t=0,κx,κz), acoustic line signal frame data iₚ(x,z) can be calculated by using Expression (7), as in Embodiment 1.

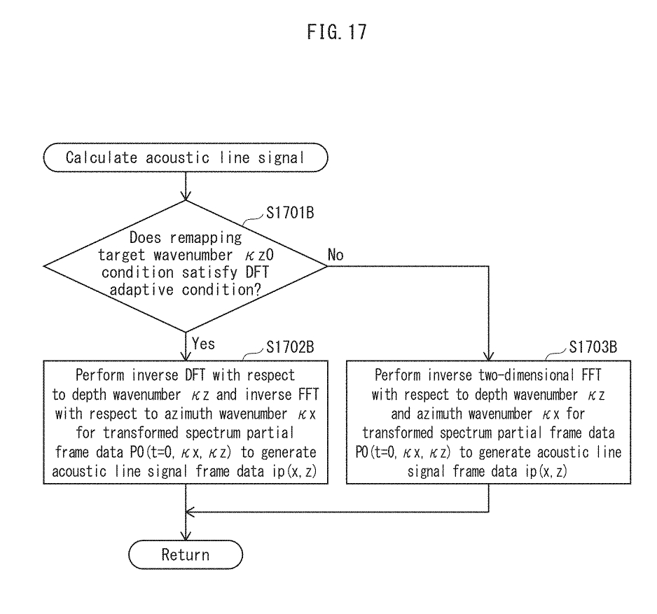

FIG. 17 is a flowchart showing detail of an operation of acoustic line signal calculation processing in step S170B.

First, it is determined by using Expressions (8) and (9) whether or not a remapping target wavenumber κz0 satisfies an adaptive condition of the inverse discrete Fourier transform (step S1701B). If not, the orthogonal space inverse transform unit 1048B performs an inverse Fourier transform on transformed spectrum partial frame data P(t=0,κx,κz) by using a fast Fourier transform on subject depth direction wavenumber κz and azimuth direction wavenumber κx to generate acoustic line signal frame data iₚ(x,z), as in Embodiment 1 (step S1703B).

On the other hand, if it is determined in step S1701B that the adaptive condition of the inverse discrete Fourier transform is satisfied, the orthogonal space inverse transform unit 1048B performs an inverse Fourier transform by using inverse discrete Fourier transform with respect to a limited target wavenumber κz0 of subject depth direction wavenumber κz and using a fast Fourier transform with respect to all of wavenumber κx of azimuth direction wavenumber κx to calculate acoustic line signal frame data iₚ(x,z) in orthogonal space (t,x) (step S1503).

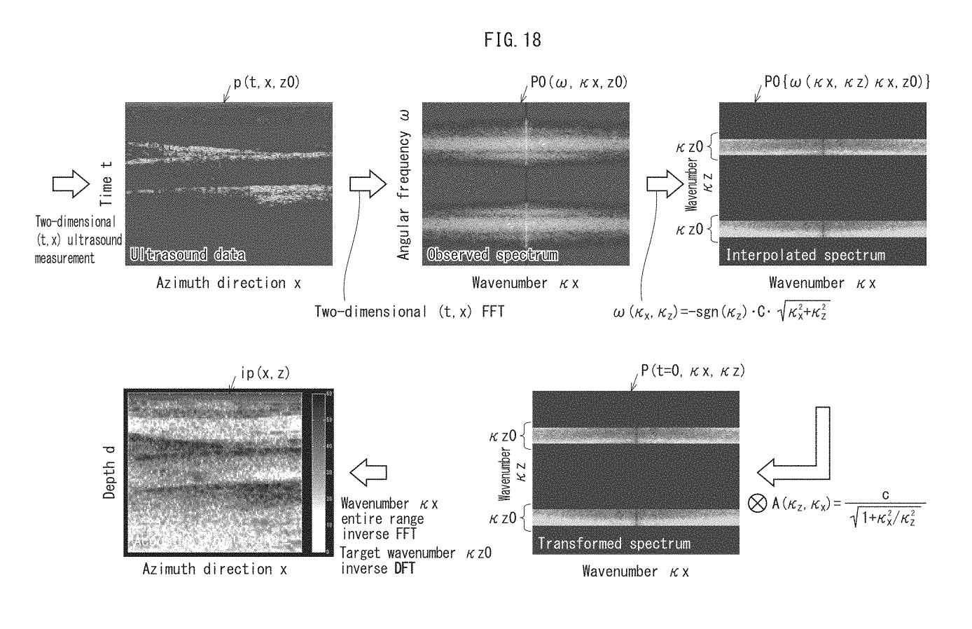

The bottom left side of FIG. 18 shows a schematic diagram of acoustic line signal frame data iₚ(x,z). In the map at the bottom left side of FIG. 18, the vertical axis represents subject depth direction z, and the horizontal axis represents azimuth direction x. As described above, in the transformed spectrum partial frame data P(t=0,κx,κz), in a limited range included in target wavenumber κz0 of depth direction wavenumber κz, a range of azimuth direction wavenumber κx included in an entire wavenumber range is calculated. Further, transformed spectrum partial frame data P (t=0,κx,κz) in third orthogonal space (κx,κz) is subject to an inverse Fourier transform by a fast Fourier transform with all of azimuth direction wavenumber κx, but even when the inverse Fourier transform is performed by using a discrete Fourier transform limited to depth direction target wavenumber κz0, focused acoustic line signal frame data iₚ(x,z) can be obtained for an entire region of subject depth direction z and azimuth direction x in orthogonal space (t,x).

Typically, in a fast Fourier transform, it is possible to calculate at high speed by setting a data number to be calculated to be a power of two. In contrast, in discrete Fourier transform, the smaller the data number to be calculated, the faster the calculation can be performed.

According to the configuration pertaining to Embodiment 3, fast Fourier transform is used for azimuth direction wavenumber κx with an entire wavenumber range as a calculation target. The number n of transducers in azimuth direction x is a power of two as described above, and therefore high speed processing can be performed by a fast Fourier transform. On the other hand, the data number to be calculated can be reduced by using a discrete Fourier transform with a range of depth direction wavenumber la included in target wavenumber κz0 as a calculation target, and therefore high speed calculation is possible.

Thus, according to the configuration pertaining to Embodiment 3, in comparison with configurations pertaining to Embodiments 1 and 2, it is possible to reduce a calculation amount even in processing of an inverse orthogonal transform to orthogonal space defined by subject depth direction and transducer row direction.

### <Summary>

According to the configuration pertaining to Embodiment 3, described above, the orthogonal space inverse transform unit 1048B, when a partial region satisfies a predefined wavenumber condition, generates acoustic line signal frame data iₚ(x,z) according to inverse discrete Fourier transform of a range corresponding to subject depth direction z target wavenumber κz0 corresponding to a partial region in transformed spectrum partial frame data P (t=0, κx,κz).

According to this configuration, it is possible to reduce an amount of calculation even in processing of inverse orthogonal transformation to an orthogonal space defined by subject depth direction and transducer row direction. Thus, it is possible to further reduce calculation in reception beamforming of reflected waves obtained from ultrasound transmission to a subject. Thus, it is possible to improve frame rate while reducing the amount of calculation in reception beamforming while suppressing deterioration of quality of an ultrasound image.

### Embodiment 4

According to the ultrasound diagnostic device 100 pertaining to Embodiment 1, the orthogonal space transform unit 1044 performs a two-dimensional Fourier transform by using fast Fourier transform on receive signal frame data p (t,x,z0) with time direction t and azimuth direction x, transforming to second orthogonal space (ω,x) of angular frequency ω and azimuth direction x to generate observed spectrum frame data P0.

However, processing methods for Fourier transform are not limited to the above, and Fourier transform may be applied to a range corresponding to target wavenumber κz0 limited by angular frequency ω of time direction t.

The following describes an ultrasound diagnostic device 100C pertaining to Embodiment 4.

### <Configuration>

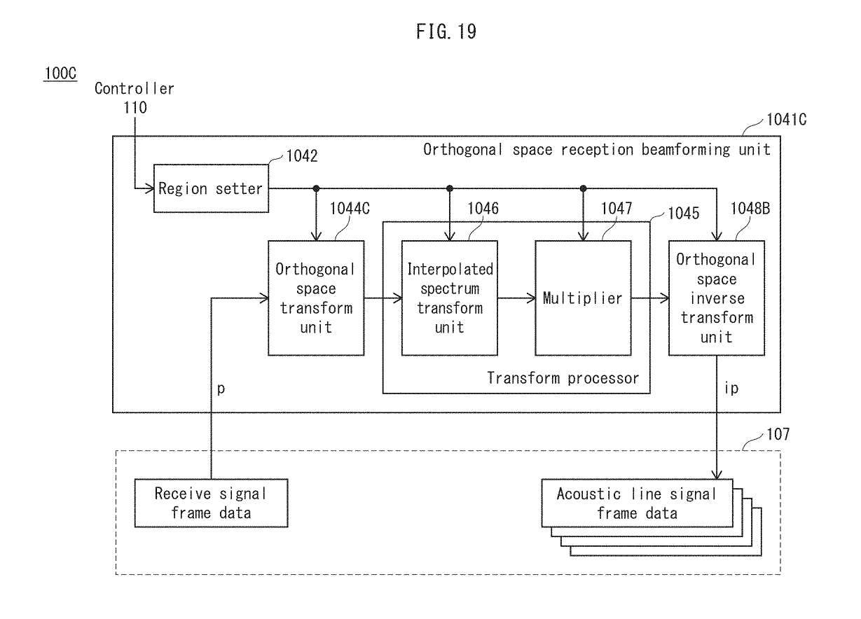

According to the ultrasound diagnostic device 100C, an orthogonal space reception beamforming unit 1041C is different from its equivalent in the ultrasound diagnostic device 100 pertaining to Embodiment 1, and this configuration is described below. Other configuration is the same as for the ultrasound diagnostic device 100 and description thereof is omitted. FIG. 19 is a function block diagram showing a configuration of the orthogonal space reception beamforming unit 1041C pertaining to Embodiment 4. In the orthogonal space reception beamforming unit 1041C, an orthogonal space transform unit 1044C and the orthogonal space inverse transform unit 104B are different from the configuration according to Embodiment 1. The orthogonal space inverse transform unit 104B is identical to that of Embodiment 3 and therefore description thereof is omitted here, while the orthogonal space transform unit 1044C is described below. Other configuration is the same as for the ultrasound diagnostic device 100 and description thereof is omitted.

The orthogonal space transform unit 1044C, regarding receive signal frame data p(t,x,z0), performs Fourier transform using discrete Fourier transform on target wavenumber κz0 of subject depth direction wavenumber κz when target wavenumber κz0 set by region setter 1042 based on operation input to the operation input unit III satisfies an adaptive condition of discrete Fourier transform. On the other hand, observed spectrum frame data P0 is generated by performing Fourier transform by using a fast Fourier transform with respect to all of wavenumber κx of azimuth direction wavenumber κx. Generated observed spectrum frame data P0 is outputted to and stored by the data storage 109.

### <Operations>

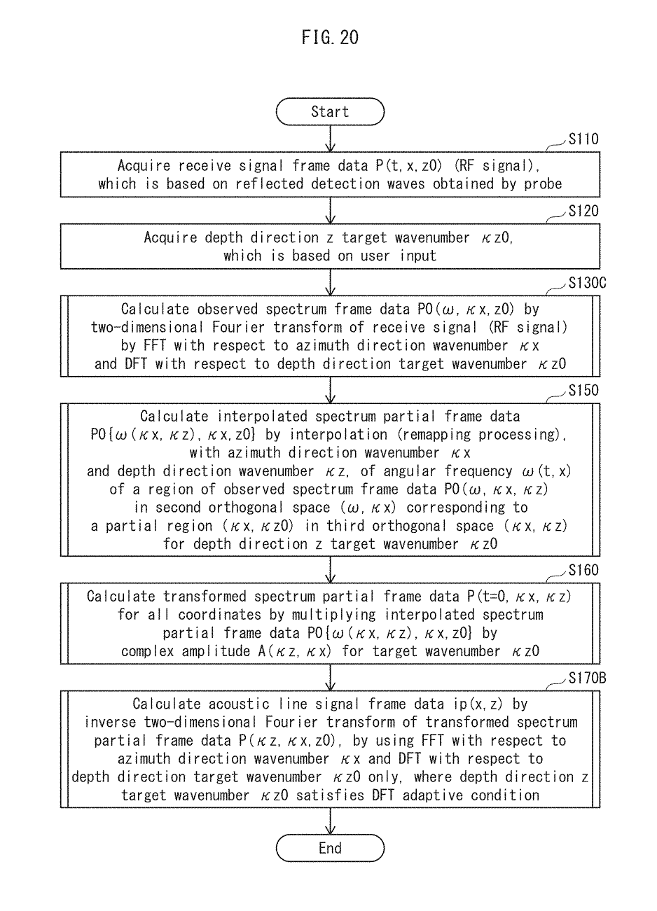

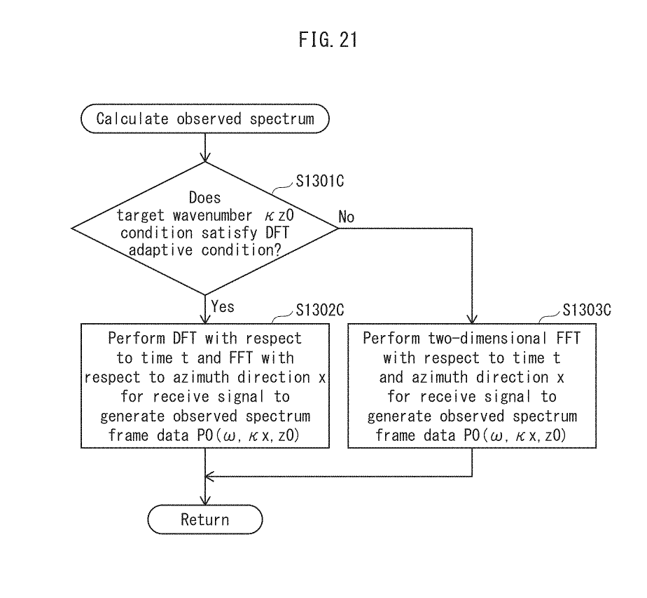

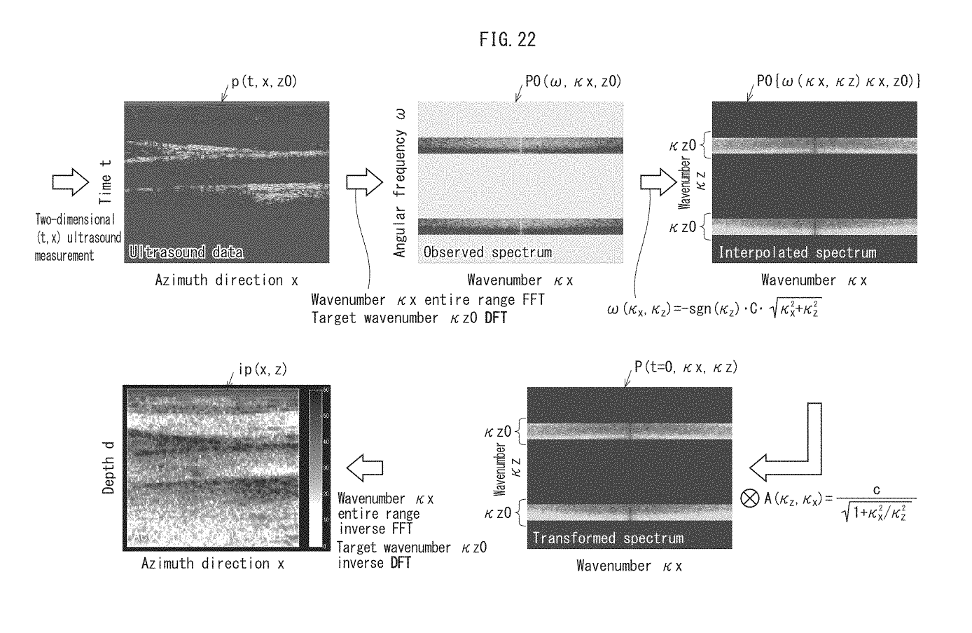

The following describes operations of the ultrasound diagnostic device 100C. FIG. 20 is a flowchart showing an overview of reception beamforming operations of the ultrasound diagnostic device 100C. In the operations of the ultrasound diagnostic device 1000, steps S130C and S170B of FIG. 20 are different from those in FIG. 6 and FIG. 7. Of these, step S170 B is the same as in FIG. 16 and therefore description is omitted here, and step S130C is described below. Further, other processing is the same as that shown in FIG. 6 and FIG. 7, and therefore description thereof is omitted here.

In step S130C in FIG. 20, the orthogonal space transform unit 1044C, with respect to receive signal frame data p(t,x,z0), performs Fourier transform using a discrete Fourier transform on a range corresponding to target wavenumber κz0, limited by time t, when the target wavenumber κz0 set based on operator input satisfies adaptive conditions of discrete Fourier transformation. On the other hand, observed spectrum frame data P0 is generated by performing a Fourier transform by using a fast Fourier transform with respect to an entire range of azimuth direction x.

FIG. 20 is a flowchart showing details of operations of calculation processing of an observed spectrum in step S130C.

First, the orthogonal space transform unit 1044C acquires depth direction z target wavenumber κz0, then determines whether or not the remapping target wavenumber κz0 satisfies an adaptive condition of discrete Fourier transform (step S1301C). When not satisfied, the orthogonal space transform unit 1044C, similarly to Embodiment 1, generates observed spectrum data P0(ω,κx,z0) by performing a Fourier transform on receive signal frame data p(t,x) by using a fast Fourier transform to time t and azimuth direction x (step S1303C).

On the other hand, when the determination in step S1301C is that the discrete Fourier transform adaptive condition is satisfied, the orthogonal space transform unit 1044C performs a Fourier transform by using a discrete Fourier transform on a range of time t corresponding to target wavenumber κz0 and performs a Fourier transform by using a fast Fourier transform on an entire range of azimuth direction x to calculate observed spectrum frame data P0(ω,κx,z0) in second orthogonal space (ω,κx) (step S1302C).

FIG. 23 is a schematic diagram showing aspects of frame data and partial frame data obtained by reception beamforming pertaining to Embodiment 4.

As described above, according to Embodiment 1, in the transformed spectrum partial frame data P(t=0,κx,κz), in a limited range included in target wavenumber κz0 of depth direction wavenumber κz, a range of azimuth direction wavenumber κx included in an entire wavenumber range is calculated.

Further, according to Embodiment 3, with respect to transformed spectrum partial frame data P(t=0,κx,κz) in third orthogonal space (κx,κz), inverse Fourier transform by using fast Fourier transform is performed on all of azimuth direction wavenumber κx, and inverse Fourier transform by using discrete Fourier transform on a limited depth direction target wavenumber κz0.

Further, according to Embodiment 4, with respect to receive signal frame data p(t,x,z0) in first orthogonal space (t,x), Fourier transform by using discrete Fourier transform is performed on a range of time t corresponding to target wavenumber κz0, and Fourier transform by using fast Fourier transform is performed on an entire range of azimuth direction x to calculate observed spectrum frame data P0(ω,κx,z0) in second orthogonal space (ω,κx). As shown at the bottom left side of FIG. 23, it can be seen that this configuration can also obtain focused acoustic line signal frame data iₚ(x,z) for an entire region of subject depth direction z and azimuth direction x in first orthogonal space (t,x).

According to the configuration pertaining to Embodiment 4, fast Fourier transform is used for azimuth direction wavenumber κx with an entire wavenumber range as calculation target. The number n of transducers in azimuth direction x is a power of two as described above, and therefore high speed processing can be performed by a fast Fourier transform. On the other hand, regarding time direction t, the data number to be calculated can be reduced by using a discrete Fourier transform with a range of depth direction wavenumber κz included in target wavenumber κz0 as a calculation target, and therefore high speed calculation is possible.

Thus, according to the configuration pertaining to Embodiment 4, when compared to the configurations of embodiments 1, 2, and 3, the amount of calculation can also be reduced in processing of orthogonal transformation from the first orthogonal space (t,x) to the second orthogonal space (ω,x).

### <Summary>

According to the configuration pertaining to Embodiment 4, described above, the orthogonal space transform unit, when a partial region satisfies a predefined wavenumber condition, performs discrete Fourier transform on a range of receive signal frame data p(t,x,z0) corresponding to a range in time direction t and a partial region of angular frequency ω, to generate observed spectrum frame data P0(ω,κx,z0).

Thus, according to this configuration, the amount of calculation can also be reduced in processing of orthogonal transformation from the first orthogonal space (t,x) to the second orthogonal space (ω,x). Thus, it is possible to further reduce calculation in reception beamforming of reflected waves obtained from ultrasound transmission to a subject. Thus, it is possible to improve frame rate while reducing the amount of calculation in reception beamforming while suppressing deterioration of quality of an ultrasound image.

### <<Modifications>>

(1) According to each embodiment and each modification, a frequency region is defined as a second orthogonal space, an orthogonal space transform unit performs Fourier transform on receive signal frame data with respect to time direction and transducer row direction, to transform to the second orthogonal space and generate observed spectrum frame data. The orthogonal space inverse transform unit performs inverse Fourier transform on transformed spectrum partial frame data with respect to subject depth direction wavenumber and transducer row direction wavenumber, to generate acoustic line signal frame data. However, a region other than a frequency region may be defined as the second orthogonal space, and for an orthogonal transform from the first orthogonal space to the second orthogonal space and an inverse orthogonal transform from the second orthogonal space to an orthogonal space defined by subject depth direction and transducer row direction, a transform other than a Fourier transform may be used. In such a case, for example, as a transform method using the second orthogonal space, a Chebyshev polynomial, a Legendre polynomial, a Hermite polynomial, principal component anaylsis, or the like, may be used.

(2) According to each embodiment and each modification, sequential processing is described such that the interpolated spectrum transform unit interpolates time direction angular frequency in observed spectrum partial frame data with transducer row direction wavenumber and subject depth direction wavenumber, to generate interpolated spectrum partial frame data; and the multiplier multiplies the interpolated spectrum partial frame data by a complex amplitude to generate transformed spectrum partial frame data. Further, according to each embodiment and each modification, processing by the interpolated spectrum transform unit and processing by the multiplier are described as being performed sequentially fir each observation point. However, processing by the interpolated spectrum transform unit and processing by the multiplier may be performed in one calculation or may be performed simultaneously.

(3) According to each embodiment and each modification, the color flow generator 1071 converts average velocity at each observation point Pij into color information to generate a color Doppler image. However, the present invention is not limited to this. For example, the velocity estimator 1053 may calculate power from a power spectrum of each observation point Pij to generate a frame power signal, and the color flow generator 1071 may convert a power value into a yellow luminance value to generate a power Doppler image.

(4) The present invention is described based on the embodiments above, but the present invention is not limited to these embodiments, and the following modifications are also included in the scope of the present invention.

For example, the present invention may be a computer system comprising a microprocessor and a memory, the memory storing a computer program and the microprocessor operating according to the computer program. For example, the present invention may be a computer system that operates (or instructs operation of connected elements) according to a computer program of a diagnostic method of an ultrasound diagnostic device of the present invention.

Further, cases in which all or part of the ultrasound diagnostic device, or all or part of a beamforming section, comprise a computer system that includes a microprocessor and a storage medium such as ROM, RAM, etc., are included in the present invention. A computer program for achieving the same operations as the devices described above may be stored in RAM or a hard disk unit. The microprocessor operates according to the computer program, thereby achieving the functions of each device.

Further, all or part of the elements of each device may be configured as one system large scale integration (LSI). A system LSI is an ultra-multifunctional LSI manufactured by integrating a plurality of elements on one chip, and more specifically is a computer system including a microprocessor, ROM, RAM, or the like. The plurality of elements can be integrated on one chip, or a portion may be integrated on one chip. Here, LSI may refer to an integrated circuit, a system LSI, a super LSI, or an ultra LSI, depending on the level of integration. A computer program for achieving the same operation as the devices described above may be stored in the RAM. The microprocessor operates according to the computer program, the system LSI thereby achieving the functions. For example, a case of the beamforming method of the present invention stored as a program of an LSI, the LSI inserted into a computer, and a predefined program (beamforming method) being executed is also included in the present invention.

Note that methods of circuit integration are not limited to LSI, and implementation may be achieved by a dedicated circuit or general-purpose processor. After LSI manufacture, a field programmable gate array (FPGA) or a reconfigurable processor, in which circuit cell connections and settings in the LSI can be reconfigured, may be used.

Further, if a circuit integration technology is introduced that replaces LSI due to advances in semiconductor technology or another derivative technology, such technology may of course be used to integrate the function blocks.

Further, all or part of the functions of an ultrasound diagnostic device pertaining to an embodiment may be implemented by execution of a program by a processor such as a CPU. All or part of the functions of an ultrasound diagnostic device pertaining to an embodiment may be implemented by a non-transitory computer-readable storage medium on which a program is stored that causes execution of a diagnostic method or beamforming method of an ultrasound diagnostic device described above. A program and signals may be recorded and transferred on a storage medium so that the program may be executed by another independent computer system, or the program may of course be distributed via a transmission medium such as the Internet.

Alternatively, each element of an ultrasound diagnostic device pertaining to an embodiment described above may be implemented by software and a programmable device such as a central processing unit (CPU), general-purpose computing on a graphic processing unit (GPGPU), a processor, or the like. The latter configuration may be referred to as general-purpose computing on a graphics processing unit (GPGPU). These elements can each be a single circuit component or an assembly of circuit components. Further, a plurality of elements can be combined into a single circuit component or can be an aggregate of a plurality of circuit components.

According to an ultrasound diagnostic device pertaining to an embodiment, the ultrasound diagnostic device includes a data storage as a storage device. However, a storage device is not limited to this, and a semiconductor memory, hard disk drive, optical disk drive, magnetic storage device, or the like may be externally connectable to the ultrasound diagnostic device.

Further, the division of function blocks in the block diagrams is merely an example, and a plurality of function blocks may be implemented as one function block, one function block may be divided into a plurality, and a portion of a function may be transferred to another function block. Further, a single hardware or software element may process the functions of a plurality of function blocks having similar functions in parallel or by time division.

Further, the order in which steps described above are executed is for illustrative purposes, and the steps may be in an order other than described above. Further, a portion of the steps described above may be executed simultaneously (in parallel) with another step.

Further, the ultrasound diagnostic device is described as having an externally connected probe and display, but may be configured with an integral probe and/or display.

Further, according to an embodiment above, the probe is configured to have a plurality of piezoelectric elements arranged in a one-dimensional direction. However, probe configuration is not limited to this example, and as further examples, a two-dimensional transducer array in which piezoelectric elements are arranged in a two-dimensional direction or a dynamic probe in which transducers arranged in a one-dimensional direction are mechanically swung to acquire a three-dimensional tomographic image may be used, and such probes may be used situationally depending on measurement. For example, when a two-dimensionally arranged probe is used it is possible to control irradiation position and direction of an ultrasound beam to be transmitted by changes to voltage application timing and value to individual piezoelectric elements.

Further, a portion of functions of the transmitter and the detection wave receiver may be included in the probe. For example, a transmission electrical signal may be generated and converted to ultrasound in the probe, based on a control signal for generating a transmission electrical signal outputted from the transmitter. It is possible to use a configuration that converts received ultrasound into a receive electric signal and generates a receive signal based on the receive electric signal in the probe.

Further, at least a portion of functions of each ultrasound diagnostic device pertaining to an embodiment, and each modification thereof, may be combined. Further, the numbers used above are all illustrative, for the purpose of explaining the present invention in detail, and the present invention is not limited to the example numbers used above.

Further, the present invention includes various modifications that are within the scope of conceivable ideas by a person skilled in the art.

### <<Summary>>

The ultrasound signal processing device pertaining to one or more embodiments is connectable to a probe in which transducers are arranged in a row, the ultrasound signal processing device including ultrasound signal processing circuitry, the ultrasound signal processing circuitry comprising: a transmission beamformer that supplies detection wave pulses to the transducers that cause the transducers to transmit detection waves that pass through at least a region of interest that represents a range to be analyzed in a subject; a reception beamformer that generates acoustic line signal frame data for observation points in the region of interest, based on reflected detection waves reflected from subject tissue and received in a time sequence by the transducers, the reflected detection waves corresponding to detection waves transmitted; and an image generator that generates ultrasound image frame data from the acoustic line signal frame data, wherein the reception beamformer includes: a receiver that acquires, for each transducer, a receive signal sequence based on reflected detection waves received in a time sequence from the subject, to generate receive signal frame data in a first orthogonal space defined by a time direction and a transducer row direction; an orthogonal space transform unit that transforms the receive signal frame data from the first orthogonal space to a second orthogonal space, to generate observed spectrum frame data; a transform processor that performs predefined calculation processing on observed spectrum partial frame data corresponding to a partial region in the second orthogonal space of the observed spectrum frame data, to generate transformed spectrum partial frame data in a third orthogonal space; and an orthogonal space inverse transform unit that performs an inverse orthogonal transform on the transformed spectrum partial frame data to an orthogonal space defined by a subject depth direction and the transducer row direction, to generate acoustic line signals for the observation points in the region of interest, in order to generate the acoustic line signal frame data.

According to this configuration, it is possible to reduce the amount of calculation in reception beamforming of a reflected wave obtained from ultrasound transmission to a subject. As a result, frame rate can be improved while suppressing deterioration in image quality.

According to another example of the configuration described above, the orthogonal space transform unit transforms the receive signal frame data into the second orthogonal space by performing a Fourier transform with respect to the time direction and the transducer row direction to generate the observed spectrum frame data, and the orthogonal space inverse transform unit transforms the transformed spectrum partial frame data by performing an inverse Fourier transform with respect to subject depth direction wavenumber and transducer row direction wavenumber to generate the acoustic line signal frame data.

According to this configuration, the receive signal frame data in the first orthogonal space can be transformed to the second orthogonal space, which is different from the first orthogonal space, to generate the observed spectrum frame data, and the transformed spectrum partial frame data in the third orthogonal space can be transformed into an orthogonal space defined by the subject depth direction and the transducer row direction, in order to generate the acoustic line signal frame data.

According to another example of any one of the configurations described above, the transform processor includes: an interpolated spectrum transform unit that interpolates time direction angular frequency in the observed spectrum partial frame data with transducer row direction wavenumber and subject depth direction wavenumber, to generate interpolated spectrum partial frame data; and a multiplier that multiplies the interpolated spectrum partial frame data by a complex amplitude to generate the transformed spectrum partial frame data.

According to this configuration, transform processing from the observed spectrum partial frame data in the second orthogonal space (ω,κx)to transformed spectrum partial frame data in the third orthogonal space (κx,κz) can be implemented, and an amount of calculation in reception beamforming can be reduced.

According to another example of any one of the configurations described above, the multiplier multiplies a range in the interpolated spectrum partial frame data corresponding to the partial region by the complex amplitude. This allows a reduction in calculation amount in reception beamforming.

According to this configuration, the observed spectrum partial frame data in the second orthogonal space (ω,κx) can be transformed to the transformed spectrum partial frame data in the third observation space (κx,κz).

According to another example of any one of the configurations above, the partial region of the observed spectrum frame data is defined by transducer row direction wavenumber and a range of time direction angular frequency.

According to this configuration, an amount of calculation can be reduced in transform processing in which the observed spectrum partial frame data in the second orthogonal space (ω,κx) is transformed to the transformed spectrum partial frame data in the third observation space (κx,κz).

According to another example of any one of the configurations above, the reception beamformer includes a region setter that sets the partial region of the observed spectrum frame data as a processing target region.

According to this configuration, a partial region to be a processing target region of the observed spectrum frame data, can be set differently based on various conditions and/or operation input.

According to another example of any one of the configurations above, the region setter determines the partial region based on band setting information that specifies a band of a range of the angular frequency inputted by a user.

According to this configuration, a user can arbitrarily set a partial region to be a processing target region of the observed spectrum frame data.

According to another example of any one of the configurations above, the region setter sets a frequency band that includes a frequency at which a maximum intensity is obtained in the observed spectrum frame data as the partial region.

According to this configuration, it is possible to set a partial region to be a processing target region based on a predefined frequency band ωa that includes a frequency at which a maximum intensity is obtained in the observed spectrum frame data P0 that is orthogonally transformed from receive signal frame data, and therefore observed spectrum partial frame data can be constructed based on a signal of a frequency at which the maximum intensity of a reflected detection wave is obtained. As a result, an amount of calculation in reception beamforming of a reflected wave obtained from ultrasound transmission into a subject can be reduced, while suppressing deterioration in ultrasound image quality. Thus, it is possible to improve frame rate while suppressing deterioration of ultrasound image quality.

According to another example of any one of the configurations above, the region setter sets, for each transducer row direction wavenumber, a frequency band that includes a frequency at which a maximum intensity is obtained in the observed spectrum frame data as the partial region.

According to this configuration, it is possible to set the frequency band that serves as a processing target region of the observed spectrum frame data differently according position along azimuth direction x, and to perform fine fitting according to position in the azimuth direction x.

According to another example of any one of the configurations above, the region setter sets a frequency range that includes a frequency at which a maximum intensity is obtained in the observed spectrum frame data as the partial region.

According to this configuration, it is possible to set a partial region to be a processing target region based on a predefined frequency range ωb that includes a frequency at which a maximum intensity is obtained in the observed spectrum frame data P0 that is orthogonally transformed from receive signal frame data, and therefore observed spectrum partial frame data can be constructed based on a signal of predefined range that exceeds one frequency band ωa, with reference to a frequency at which the maximum intensity of a reflected detection wave is obtained.

According to another example of any one of the configurations above, the region setter sets, for each transducer row direction wavenumber, a frequency range that includes a frequency at which a maximum intensity is obtained in the observed spectrum frame data as the partial region.

According to this configuration, it is possible to set the frequency range that serves as a processing target region of the observed spectrum frame data differently according position along azimuth direction x, and to perform fine fitting according to position in the azimuth direction x.

According to another example of any one of the configurations above, the region setter sets the partial region for every predefined number of transmission times of detection waves.

According to this configuration, it is possible to set a different partial region to be a processing target of the observed spectrum frame data for each transmission event, and to perform fine fitting according to each transmission event.

According to another example of any one of the configurations above, the orthogonal space inverse transform unit, when the partial region satisfies a predefined wavenumber condition, generates the acoustic line signal frame data by performing an inverse discrete Fourier transform on a range of the transformed spectrum partial frame data corresponding to a wavenumber range in the subject depth direction corresponding to the partial region.

According to this configuration, in contrast to configurations pertaining to Embodiments 1 and 2, it is possible to reduce a calculation amount even in processing of an inverse orthogonal transform to orthogonal space defined by subject depth direction and transducer row direction. Thus, it is possible to further reduce calculation in reception beamforming of reflected waves obtained from ultrasound transmission to a subject.

According to another example of any one of the configurations above, the orthogonal space transform unit, when the partial region satisfies a predefined wavenumber condition, generates the observed spectrum frame data by performing discrete Fourier transform on a range of the partial region corresponding to a range in the time direction corresponding to angular frequency of the partial region.

According to this configuration, in contrast to the configurations of Embodiments 1, 2, and 3, the amount of calculation can also be reduced in processing of orthogonal transformation from the first orthogonal space (t,x) to the second orthogonal space (ω,x). Thus, it is possible to further reduce calculation in reception beamforming of reflected waves obtained from ultrasound transmission to a subject.

According to another example of any one of the configurations above, the transmission beamformer transmits an unfocused ultrasound beam that is not focused on a point in the subject.

According to this configuration, in an ultrasound signal processing method of the present disclosure, plane wave transmission and reception is preferred for improving frame rate, and therefore by transmitting a plane wave as a detection wave, it is possible to further improve frame rate while reducing the amount of calculation in reception beamforming while suppressing deterioration of quality of an ultrasound image.

According to another example of any one of the configurations above, the image generator generates the ultrasound image frame data based on phase information of the acoustic line signal frame data.

According to this configuration, application of acoustic line signal output to CFM signal, when compared to application to a B mode tomographic image, has less ultrasound image quality deterioration while limiting angular frequency of observed spectrum frame data.

An ultrasound signal processing method pertaining to one or more embodiments is an ultrasound signal processing method of an ultrasound signal processing device that is connectable to a probe in which transducers are arranged in a row, the ultrasound signal processing method comprising: supplying detection wave pulses to the transducers that cause the transducers to transmit detection waves that pass through at least a region of interest that represents a range to be analyzed in a subject; generating acoustic line signal frame data for observation points in the region of interest, based on reflected detection waves reflected from subject tissue and received in a time sequence by the transducers, the reflected detection waves corresponding to detection waves transmitted; and generating ultrasound image frame data from the acoustic line signal frame data, wherein the generating of the acoustic line signal frame data includes: acquiring, for each transducer, a receive signal sequence based on reflected detection waves received in a time sequence from the subject, to generate receive signal frame data in a first orthogonal space defined by a time direction and a transducer row direction; transforming the receive signal frame data from the first orthogonal space to a second orthogonal space, to generate observed spectrum frame data; performing predefined calculation processing on observed spectrum partial frame data corresponding to a partial region in the second orthogonal space of the observed spectrum frame data, to generate transformed spectrum partial frame data in a third orthogonal space; and performing an inverse orthogonal transform on the transformed spectrum partial frame data to an orthogonal space defined by a subject depth direction and the transducer row direction, to generate acoustic line signals for the observation points in the region of interest, in order to generate the acoustic line signal frame data.

According to another example of the method described above, the generating of the observed spectrum frame data includes transforming the receive signal frame data into the second orthogonal space by performing a Fourier transform with respect to the time direction and the transducer row direction to generate the observed spectrum frame data, and the generating of the acoustic line signal frame data includes transforming the transformed spectrum partial frame data by performing an inverse Fourier transform with respect to subject depth direction wavenumber and transducer row direction wavenumber to generate the acoustic line signal frame data.

According to another example of the method above, the generating of the transformed spectrum partial frame data includes: interpolating time direction angular frequency in the observed spectrum partial frame data with transducer row direction wavenumber and subject depth direction wavenumber, to generate interpolated spectrum partial frame data; and multiplying the interpolated spectrum partial frame data by a complex amplitude to generate the transformed spectrum partial frame data.

As described above, according to the ultrasound signal processing device, ultrasound signal processing method, and ultrasound diagnostic device using same, each pertaining to an aspect of the present disclosure, it is possible to reduce an amount of calculation in reception beamforming of reflected waves obtained from ultrasound transmission into a subject. Thus, particularly when performing signal processing using phase information, it is possible to improve frame rate while suppressing degradation of quality, such as degradation of quality of blood flow information.

Although the present invention has been fully described by way of examples with reference to the accompanying drawings, it is to be noted that various changes and modifications will be apparent to those skilled in the art. Therefore, unless such changes and modifications depart from the scope of the present invention, they should be construed as being included therein.

What is claimed is:

1. An ultrasound signal processing device that is connectable to a probe in which transducers are arranged in a row, the ultrasound signal processing device comprising:
ultrasound signal processing circuitry comprising:
a transmission beamformer that supplies detection wave pulses to the transducers that cause the transducers to transmit detection waves that pass through at least a region of interest that represents a range to be analyzed in a subject;
a reception beamformer that generates acoustic line signal frame data for observation points in the region of interest, based on reflected detection waves reflected from subject tissue and received in a time sequence by the transducers, the reflected detection waves corresponding to detection waves transmitted; and
an image generator that generates ultrasound image frame data from the acoustic line signal frame data, wherein
the reception beamformer includes:
a receiver that acquires, for each transducer, a receive signal sequence based on reflected detection waves received in a time sequence from the subject, to generate receive signal frame data in a first orthogonal space defined by a time direction and a transducer row direction;
an orthogonal space transform unit that transforms the receive signal frame data from the first orthogonal space to a second orthogonal space, to generate observed spectrum frame data;
a transform processor that performs predefined calculation processing on observed spectrum partial frame data corresponding to a partial region in the second orthogonal space of the observed spectrum frame data, to generate transformed spectrum partial frame data in a third orthogonal space; and
an orthogonal space inverse transform unit that performs an inverse orthogonal transform on the transformed spectrum partial frame data to an orthogonal space defined by a subject depth direction and the transducer row direction, to generate acoustic line signals for the observation points in the region of interest, in order to generate the acoustic line signal frame data.

2. The ultrasound signal processing device of claim 1, wherein
the orthogonal space transform unit transforms the receive signal frame data into the second orthogonal space by performing a Fourier transform with respect to the time direction and the transducer row direction to generate the observed spectrum frame data, and
the orthogonal space inverse transform unit transforms the transformed spectrum partial frame data by performing an inverse Fourier transform with respect to subject depth direction wavenumber and transducer row direction wavenumber to generate the acoustic line signal frame data.

3. The ultrasound signal processing device of claim 2, wherein
the transform processor:
interpolates time direction angular frequency in the observed spectrum partial frame data with transducer row direction wavenumber and subject depth direction wavenumber, to generate interpolated spectrum partial frame data; and
multiplies the interpolated spectrum partial frame data by a complex amplitude to generate the transformed spectrum partial frame data.

4. The ultrasound signal processing device of claim 3, wherein
the transform processor multiplies a range in the interpolated spectrum partial frame data corresponding to the partial region by the complex amplitude.

5. The ultrasound signal processing device of claim 2, wherein
the partial region of the observed spectrum frame data is defined by transducer row direction wavenumber and a range of time direction angular frequency.

6. The ultrasound signal processing device of claim 1, wherein
the reception beamformer includes a region setter that sets the partial region of the observed spectrum frame data as a processing target region.

7. The ultrasound signal processing device of claim 6, wherein
the region setter determines the partial region based on band setting information that specifies a band of a range of the angular frequency inputted by a user.

8. The ultrasound signal processing device of claim 6, wherein
the region setter sets a frequency band that includes a frequency at which a maximum intensity is obtained in the observed spectrum frame data as the partial region.

9. The ultrasound signal processing device of claim 8, wherein
the region setter sets, for each transducer row direction wavenumber, a frequency band that includes a frequency at which a maximum intensity is obtained in the observed spectrum frame data as the partial region.

10. The ultrasound signal processing device of claim 6, wherein
the region setter sets a frequency range that includes a frequency at which a maximum intensity is obtained in the observed spectrum frame data as the partial region.

11. The ultrasound signal processing device of claim 10, wherein
the region setter sets, for each transducer row direction wavenumber, a frequency range that includes a frequency at which a maximum intensity is obtained in the observed spectrum frame data as the partial region.

12. The ultrasound signal processing device of claim 6, wherein
the region setter sets the partial region for every predefined number of transmission times of detection waves.

13. The ultrasound signal processing device of claim 2, wherein
the orthogonal space inverse transform unit, when the partial region satisfies a predefined wavenumber condition, generates the acoustic line signal frame data by performing an inverse discrete Fourier transform on a range of the transformed spectrum partial frame data corresponding to a wavenumber range in the subject depth direction corresponding to the partial region.

14. The ultrasound signal processing device of claim 2, wherein
the orthogonal space transform unit, when the partial region satisfies a predefined wavenumber condition, generates the observed spectrum frame data by performing discrete Fourier transform on a range of the partial region corresponding to a range in the time direction corresponding to angular frequency of the partial region.

15. The ultrasound signal processing device of claim 1, wherein
the transmission beamformer transmits an unfocused ultrasound beam that is not focused on a point in the subject.

16. The ultrasound signal processing device of claim 1, wherein
the image generator generates the ultrasound image frame data based on phase information of the acoustic line signal frame data.

17. An ultrasound signal processing method of an ultrasound signal processing device that is connectable to a probe in which transducers are arranged in a row, the ultrasound signal processing method comprising:
supplying detection wave pulses to the transducers that cause the transducers to transmit detection waves that pass through at least a region of interest that represents a range to be analyzed in a subject;
generating acoustic line signal frame data for observation points in the region of interest, based on reflected detection waves reflected from subject tissue and received in a time sequence by the transducers, the reflected detection waves corresponding to detection waves transmitted; and
generating ultrasound image frame data from the acoustic line signal frame data, wherein
the generating of the acoustic line signal frame data includes:
acquiring, for each transducer, a receive signal sequence based on reflected detection waves received in a time sequence from the subject, to generate receive signal frame data in a first orthogonal space defined by a time direction and a transducer row direction;
transforming the receive signal frame data from the first orthogonal space to a second orthogonal space, to generate observed spectrum frame data;
performing predefined calculation processing on observed spectrum partial frame data corresponding to a partial region in the second orthogonal space of the observed spectrum frame data, to generate transformed spectrum partial frame data in a third orthogonal space; and
performing an inverse orthogonal transform on the transformed spectrum partial frame data to an orthogonal space defined by a subject depth direction and the transducer row direction, to generate acoustic line signals for the observation points in the region of interest, in order to generate the acoustic line signal frame data.

18. The ultrasound signal processing method of claim 17, wherein
the generating of the observed spectrum frame data includes transforming the receive signal frame data into the second orthogonal space by performing a Fourier transform with respect to the time direction and the transducer row direction to generate the observed spectrum frame data, and
the generating of the acoustic line signal frame data includes transforming the transformed spectrum partial frame data by performing an inverse Fourier transform with respect to subject depth direction wavenumber and transducer row direction wavenumber to generate the acoustic line signal frame data.

19. The ultrasound signal processing method of claim 18, wherein
the generating of the transformed spectrum partial frame data includes:
interpolating time direction angular frequency in the observed spectrum partial frame data with transducer row direction wavenumber and subject depth direction wavenumber, to generate interpolated spectrum partial frame data; and
multiplying the interpolated spectrum partial frame data by a complex amplitude to generate the transformed spectrum partial frame data.
